SEQUENCE LISTING (10 sequences)

SEQ ID NO: 1 - protein, 796 aa, Homo sapiens
MPTTQQSPQDEQEKLLDEAIQAVKVQSFQMKRCLDKNKLMDALKHASNMLGELRTSMLSPKSYYELYMAISDELHYLEVYLTDEFAKGRKVADLYELVQYAGNIIPRLYLLITVGVVYVKSFPQSRKDILKDLVEMCRGVQHPLRGLFLRNYLLQCTRNILPDEGEPTDEETTGDISDSMDFVLLNFAEMNKLWVRMQHQGHSRDREKRERERQELRILVGTNLVRLSQLEGVNVERYKQIVLTGILEQVVNCRDALAQEYLMECIIQVFPDEFHLQTLNPFLRACAELHQNVNVKNIIIALIDRLALFAHREDGPGIPADIKLFDIFSQQVATVIQSRQDMPSEDVVSLQVSLINLAMKCYPDRVDYVDKVLETTVEIFNKLNLEHIATSSAVSKELTRLLKIPVDTYNNILTVLKLKHFHPLFEYFDYESRKSMSCYVLSNVLDYNTEIVSQDQVDSIMNLVSTLIQDQPDQPVEDPDPEDFADEQSLVGRFIHLLRSEDPDQQYLILNTARKHFGAGGNQRIRFTLPPLVFAAYQLAFRYKENSKVDDKWEKKCQKIFSFAHQTISALIKAELAELPLRLFLQGALAAGEIGFENHETVAYEFMSQAFSLYEDEISDSKAQLAAITLIIGTFERMKCFSEENHEPLRTQCALAASKLLKKPDQGRAVSTCAHLFWSGRNTDKNGEELHGGKRVMECLKKALKIANQCMDPSLQVQLFIEILNRYIYFYEKENDAVTIQVLNQLIQKIREDLPNLESSEETEQINKHFHNTLEHLRLRRESPESEGPIYEGLIL

SEQ ID NO: 2 - protein, 327 aa, Homo sapiens
MSFLGGFFGPICEIDIVLNDGETRKMAEMKTEDGKVEKHYLFYDGESVSGKVNLAFKQPGKRLEHQGIRIEFVGQIELFNDKSNTHEFVNLVKELALPGELTQSRSYDFEFMQVEKPYESYIGANVRLRYFLKVTIVRRLTDLVKEYDLIVHQLATYPDVNNSIKMEVGIEDCLHIEFEYNKSKYHLKDVIVGKIYFLLVRIKIQHMELQLIKKEITGIGPSTTTETETIAKYEIMDGAPVKGESIPIRLFLAGYDPTPTMRDVNKKFSVRYFLNLVLVDEEDRRYFKQQEIILWRKAPEKLRKQRTNFHQRFESPESQASAEQPEM

SEQ ID NO: 3 - protein, 336 aa, Homo sapiens
MSFFGFGQSVEVEILLNDAESRKRAEHKTEDGKKEKYFLFYDGETVSGKVSLALKNPNKRLEHQGIKIEFIGQIELYYDRGNHHEFVSLVKDLARPGEITQSQAFDFEFTHVEKPYESYTGQNVKLRYFLRATISRRLNDVVKEMDIVVHTLSTYPELNSSIKMEVGIEDCLHIEFEYNKSKYHLKDVIVGKIYFLLVRIKIKHMEIDIIKRETTGTGPNVYHENDTIAKYEIMDGAPVRGESIPIRLFLAGYELTPTMRDINKKFSVRYYLNLVLIDEEERRYFKQQEVVLWRKGDIVRKSMSHQAAIASQRFEGTTSLGEVRTPSQLSDNNCRQ

SEQ ID NO: 4 - DNA, 2391 bp, Mus musculus
atgcctacaacacagcagtcaccccaggatgagcaggaaaaacttttggatgaagccatccaggctgtgaaggttcagtcattccagatgaaaagatgcctggacaaaaacaagctgatggatgctctgaagcatgcctccaatatgcttggagagctccggacctctatgctgtcaccaaagagttactatgaactttatatggctatttctgatgaactgcactacttggaggtctacctgactgatgaatttgctaaaggaagaaaggtggcagatctctatgaacttgtacagtacgcgggaaacattattccaaggctttatctcttgatcacagttggagttgtatatgtcaagtcatttcctcaatccaggaaagatattttgaaagatttggtagaaatgtgccgtggtgtgcagcatccgctaaggggtttgtttcttcgaaattaccttcttcagtgtactaggaacattttacctgatgaaggagagccaacagatgaagaaacaactggtgatatcagtgattccatggattttgtactactcaactttgcagaaatgaataagctctgggtgcggatgcagcatcaaggacatagtcgagatagagaaaaaagagaacgagagagacaagaactgagaattttagtgggaacaaatttggtgcgccttagtcagttggaaggtgtaaatgtggaacgttacaaacagattgttctaacaggcattctggagcaggttgtgaattgtagggatgctctggctcaggagtatctcatggagtgcatcattcaggtttttcctgatgagtttcatcttcagactttgaatccttttcttagagcttgtgctgaattacaccaaaatgtaaatgtgaaaaacataatcattgctttaattgacagattagctttatttgctcatcgtgaagatggacccggaattccagctgagattaaactttttgacatattttcacaacaggtggctacagtcatacagtccagacaagacatgccatcagaggatgttgtatctttacaagtctctctcattaatcttgctatgaaatgttaccctgatcgtgtggactacgtcgataaagttctagaaacaacagtggagatattcaataagcttaaccttgaacatattgctaccagtagtgcagtttcaaaggagcttaccagacttttgaagatacctgttgatacttacaacaatatcttaacagtattaaagttaaagcatttccacccactttttgagtactttgactacgagtccagaaagagcatgagctgttatgtgcttagtaatgttctggattataacacagaaatcgtctctcaggaccaggtagattccataatgaatttggtgtccacattgattcaggatcagccagaccaacctgtagaagaccctgatccagaagacttcgctgatgaacagagccttgttggccgatttattcatctgctacgttctgatgatcctgaccagcagtatttgattttgaatacggcacgaaaacattttggggctggtggaaatcagcggattcgcttcacactgccacctttggtatttgcagcttaccagttggcttttcgatacaaagagaattcccaaatggatgacaagtgggaaaagaaatgccagaagatattttcatttgctcaccagactatcagtgctttgattaaagctgagctggctgaattaccactgagactttttcttcaaggagcattagctgctggagaaattggctttgaaaatcatgaaacagtagcatatgaatttatgtcccaggcattttctctatatgaagatgaaatcagtgattctaaagcacagctggctgctatcaccttgatcatcggtacttttgagaggatgaaatgcttcagtgaagagaatcatgaacccttgagaactcagtgtgcacttgctgcatcaaaacttctgaaaaaaccagatcaaggccgagccgtgagcacatgtgcgcatctcttttggtctggccgaaacacagacaaaaatggggaagagcttcatggaggtaaaagggtcatggagtgcctaaagaaggcactaaaaatagcaaatcagtgcatggacccctctctacaagttcagctctttatagagattctgaacaggtacatctatttctatgaaaaagaaaatgatgcggtaaccattcaagtcttgaaccaacttattcaaaagattcgagaagatctcccaaatcttgagtccagtgaagaaacagaacaaataaacaagcattttcacaacacattggagcacttgcgctcaagacgggaatcaccagagtctgaggggccaatctatgaaggtctcatcctttaa

SEQ ID NO: 5 - DNA, 984 bp, Mus musculus
atgagttttcttggaggcttttttggtcccatttgtgagattgatgttgcccttaatgatggggaaaccaggaaaatggcagaaatgaaaactgaggatggcaaagtagaaaaacactatctcttctatgatggcgagtctgtctcaggaaaggtaaacctagcctttaagcagcctggaaagaggctagagcatcaaggaattagaattgaatttgtaggtcaaattgagcttttcaatgacaagagtaatactcatgaatttgtaaacctagtgaaggaactagccttgcctggagaactgactcagagcagaagctatgactttgaatttatgcaagttgaaaagccatatgagtcatacatcggtgccaatgtccgcctgaggtattttcttaaggtgacaattgtgagaagattgacagacttagtgaaagagtacgatcttattgttcatcagctagccacctatcctgatgtcaacaactctattaaaatggaagtgggcattgaagactgtctgcacatagagtttgaatataataagtccaagtatcatttaaaggatgtaattgttggaaaaatttacttcttattagtaagaataaaaatacaacacatggaattacagctgatcaagaaagagatcacaggaattggacccagcaccacaacagagacagaaacaatcgctaagtatgaaataatggatggggcgccagtaaaaggagaatctattccgataagattgttcttagcagggtatgacccaacccccacgatgagagatgtgaacaagaagttttcagtaaggtactttctaaacctcgtgcttgttgatgaggaggaccgaaggtacttcaagcagcaggagatcatcctgtggagaaaagcacccgagaaactgagaaaacagaggacgaactttcaccagcggtttgaatctccagactcgcaggcctctgcggagcagcctgagatgtaa

SEQ ID NO: 6 - DNA, 1011 bp, Mus musculus
atgagcttcttcggcttcgggcagagcgtggaggtggaaatcttgctgaatgatgcggagagtaggaagcgagcggagcacaagactgaggacgggaagaaggagaaatatttcctcttctacgacggggaaaccgtctcggggaaagtgagcctatcactgaagaaccccaacaagcgactagagcaccagggcatcaagatcgagttcattgggcagatcgaactctactatgaccgtgggaaccaccatgagtttgtgtctttggtgaaggacctggctcggccaggagagatcacccaatcgcaggccttcgactttgagttcacccatgtggaaaagccgtatgaatcctacacaggacagaatgtgaagctccgctatttccttcgagccaccatcagccgccgcctcaatgatgttgttaaagagatggacattgtagttcacacacttagcacataccccgagctgaactcatccatcaagatggaagttggcattgaggattgcctgcacattgaatttgagtacaacaaatccaaataccacttgaaagatgtcattgtagggaagatatacttcctgctggtgagaatcaagatcaagcacatggagatagacatcatcaaacgagagacaacaggcacgggtcccaacgtgtaccacgagaacgacacaatagcgaagtatgagatcatggacggggcaccagtccgaggtgagtccatccccatcaggctcttcctggcagggtatgagctcacacccaccatgcgtgacataaataagaagttctctgtgcgctattacctcaacttggtgctgatagatgaggaggaacggcgctacttcaagcagcaggaagtggtgttgtggcggaagggtgacatcgtacggaagagcatgtcccaccaggcagccattgcctcacagcgcttcgagggcacaacctccctgggtgaggtgcggacccctggccaactgtctgacaacaacagcaggcagtag

SEQ ID NO: 7 - DNA, 2391 bp, Mus musculus
atgcccacaacacagcagagcccacaggatgaacaggaaaagctgctggatgaggccatccaggctgtgaaagtgcagtccttccagatgaaaagatgtctggataagaataagctgatggacgccctgaagcatgccagcaacatgctgggcgagctgaggaccagcatgctgtctcccaagtcctactacgagctgtacatggccatttctgacgagctgcactacctggaggtgtacctcacagatgagttcgccaaggggagaaaggtggccgatctgtatgagctggtgcagtatgctggcaacatcatccccaggctgtacctgctgatcaccgtgggcgtggtgtatgtgaagagcttcccccagtctagaaaggatatcctgaaggacctggtggaaatgtgcaggggagtgcagcatcctctgagaggcctgtttctgagaaactacctgctgcagtgtaccaggaatatcctgcccgacgaaggcgagcctactgatgaagaaacaaccggcgacatcagcgacagcatggattttgtgctgctgaacttcgccgagatgaacaaactgtgggtgagaatgcagcaccagggacacagcagggacagagagaagagagagagggagagacaggagctgagaatcctggtgggcaccaacctggtgaggctgtcccagctggagggcgtgaatgtggagagatataagcagattgtgctgacaggaatcctggagcaggtggtgaactgcagagatgccctggcccaggagtatctgatggaatgcatcattcaggtgttcccagatgagttccacctgcagacactgaaccctttcctgagggcctgcgccgagctgcatcagaacgtgaacgtcaagaacatcatcattgctctgattgacaggctggccctgtttgcccatagagaggatggacctggcatcccagccgagatcaagctgtttgacattttcagccagcaggtggccaccgtgatccagtctaggcaggacatgccttccgaagatgtggtgagcctgcaggtgtccctgatcaatctggccatgaagtgttaccctgatagagtggattacgtggacaaggtgctggagaccacagtggagatcttcaacaagctgaacctggagcacatcgccacttcttctgctgtgtctaaagaactgacaagactgctgaagatccctgtggacacctacaacaacattctgaccgtgctgaagctgaaacacttccatccactgttcgagtactttgattatgagagcaggaagtccatgagctgctacgtgctgagcaatgtgctggactacaatacagaaatcgtgtcccaggatcaggtggacagcatcatgaatctggtgagcacactgatccaggaccagcctgatcagccagtggaggaccccgaccctgaggacttcgctgacgagcagagcctggtgggcaggttcatccacctgctgaggtctgatgatccagaccagcagtacctgatcctgaacaccgccaggaagcactttggagccggaggcaaccagaggatcagattcaccctgcctcccctggtgtttgccgcctaccagctggctttcagatacaaggaaaattcccagatggatgacaagtgggagaaaaaatgccagaagatcttttcttttgctcaccagaccatctctgccctgatcaaagctgagctggctgaactgccactgagactgttcctccagggggccctggctgccggggagatcggctttgagaaccatgagactgtggcctatgaattcatgtctcaggccttctctctgtacgaagatgagatcagcgacagcaaggctcagctggctgctatcacactgatcattggaacatttgaaaggatgaagtgcttctccgaggaaaaccacgagccactgagaacacagtgtgcactggccgcctccaagctgctgaagaagcctgaccagggcagagccgtgtctacctgtgcccacctgttctggagcggaagaaacacagacaaaaatggagaggagctgcatggcggcaagagggtgatggagtgcctgaagaaagccctgaagattgccaaccagtgcatggacccaagcctgcaggtgcagctgttcatcgagatcctgaacaggtacatctacttctacgagaaggagaatgacgccgtgaccattcaggtgctgaatcagctgatccagaaaatcagggaagacctgcccaacctggagagctctgaagagacagagcagatcaacaagcattttcacaacactctggaacatctgaggtccaggagggagtccccagagtctgagggacccatctatgaggggctgatcctgtga

SEQ ID NO: 8 - DNA, 984 bp, Mus musculus
atgagcttcctgggcggcttcttcggccccatctgtgagatcgacgtggccctgaacgacggcgagaccagaaagatggccgagatgaagacagaggatggcaaggtggagaagcactacctgttctacgacggagagtctgtgtccggcaaggtgaacctggccttcaagcagcctgggaagaggctggagcaccagggcatcagaatcgagttcgtgggccagatcgagctgttcaacgacaagagcaacacccacgagtttgtgaacctggtgaaggagctggctctgcctggcgagctgacccagagcagaagctacgacttcgagttcatgcaggtggagaagccttacgagagctacatcggcgccaacgtgagactgagatacttcctgaaggtgaccatcgtgaggagactgaccgacctggtgaaggagtatgacctgatcgtgcaccagctggccacctaccctgacgtgaacaacagcatcaagatggaggtgggcatcgaggactgcctgcacatcgagttcgagtacaacaagtccaagtaccacctgaaggacgtgatcgtgggcaagatctacttcctgctggtgaggatcaagatccagcacatggagctgcagctgatcaagaaggagatcaccggcatcggcccttccacaaccaccgagacagagacaatcgccaagtacgagatcatggacggcgcccctgtgaagggcgagagcatccctatcaggctgttcctggccggctacgaccctacccctaccatgagagacgtgaacaagaagttcagcgtgaggtacttcctgaacctggtgctggtggacgaggaggacagaagatacttcaagcagcaggagatcatcctgtggaggaaggcccctgagaagctgaggaagcagaggaccaacttccaccagagattcgagtcccctgacagccaggccagcgccgagcagccagagatgtga

SEQ ID NO: 9 - DNA, 1011 bp, Mus musculus
atgagtttttttgggtttggacagtcagtggaggtggagatcctgctgaacgacgccgagagcaggaagagggccgagcacaagacagaggatggcaagaaggagaagtacttcctgttctacgacggagagaccgtgagcggcaaggtgtccctgagcctgaagaacccaaacaagagactggagcaccagggcatcaagatcgagttcatcggccagatcgagctgtactacgacaggggcaaccaccacgagttcgtgagcctggtgaaggacctggccagacctggcgagatcacccagagccaggccttcgacttcgagttcacccacgtggagaagccttacgagagctacacaggccagaacgtgaagctgaggtacttcctgagagccaccatcagcagaagactgaacgacgtggtgaaggagatggacatcgtggtgcacaccctgagcacctaccctgagctgaactcttccatcaagatggaggtgggcatcgaggactgcctgcacatcgagttcgagtacaacaagagcaagtaccacctgaaggacgtgatcgtgggcaagatctacttcctgctggtgagaatcaagatcaagcacatggagatcgacatcatcaagagagagaccacaggcacaggccctaacgtgtaccacgagaacgacaccatcgccaagtacgagatcatggacggcgcccctgtgagaggcgagagcatccctatcaggctgttcctggccggctacgagctgacccctaccatgagggacatcaacaagaagttcagcgtgagatactacctgaacctggtgctgatcgatgaggaggagagaagatacttcaagcagcaggaggtggtgctgtggagaaagggcgacatcgtgagaaagagcatgagccaccaggccgccatcgccagccagagattcgagggcaccaccagcctgggcgaggtgaggacccctggccagctgagcgacaacaacagcagacagtga

SEQ ID NO: 10 - DNA, 2391 bp, Homo sapiens
atgcctacaacacagcagtcccctcaggatgagcaggaaaagctcttggatgaagccatacaggctgtgaaggtccagtcattccaaatgaagagatgcctggacaaaaacaagctcatggatgctctaaaacatgcttctaatatgcttggtgaactccggacttctatgttatcaccaaagagttactatgaactttatatggccatttctgatgaactgcactacttggaggtctacctgacagatgagtttgctaaaggaaggaaagtggcagatctctacgaacttgtacagtatgctggaaacattatcccaaggctttaccttctgatcacagttggagttgtatatgtcaagtcatttcctcagtccaggaaggatattctgaaagatttggtagaaatgtgccgtggtgtgcaacatcccttgaggggtctgtttcttcgaaattaccttcttcagtgtaccagaaatatcttacctgatgaaggagagccaacagatgaagaaacaactggtgacatcagtgattccatggatttcgtactgctcaactttgcagaaatgaacaagctctgggtgcgaatgcagcatcagggacatagccgagatagagaaaaaagagaacgagaaagacaagaactgaggattctagtgggaacaaatttggtgcgcctcagtcagttggaaggtgtaaatgtggaacgttacaaacagattgttctgactggcatattggagcaagttgtaaactgtagggatgctttggctcaagaatatctcatggagtgtattattcaggtattccctgatgaatttcacctccagactttgaatcctttccttcgggcctgtgctgagttacaccagaatgtaaatgtgaagaacataatcattgctttaattgatagattagctttatttgctcaccgtgaagatggacctggaatcccagcggatattaaactattcgatatattctcacagcaggtggctacagtgatacagtcaagacaagacatgccttcagaggatgttgtatctttacaagtatctcttattaatcttgccatgaaatgttaccctgatcgtgtggactatgttgataaagtactagaaacaacagtggagatattcaataagctcaaccttgaacatattgctaccagtagtgcagtttcaaaggaactcaccagactattgaaaataccagttgacacttacaacaatatattaacagtcttgaaattaaaacatttccacccactctttgagtactttgactacgagtccagaaagagcatgagttgttatgtgcttagtaatgttctggattataacacagaaattgtatctcaagaccaggtggattccataatgaatttggtatccacgttgattcaagatcagccagatcaacctgtagaagaccctgatccagaagatttcgctgatgagcagagccttgtgggccgcttcattcatctgctgcgctctgaggaccctgaccagcagtacttgatattgaacacagcacgaaaacatttcggagctggtggaaatcagcggattcgcttcacactgccacctttggtatttgcagcttaccagctggcatttcgatataaagagaactctaaagtggatgacaaatgggaaaagaaatgccagaagatattctcatttgcccaccagactatcagtgctttgatcaaagcagagctggcagaattgccgttaagactattccttcaaggagcactagctgctggggaaattggatttgaaaatcatgaaacagtcgcatatgaattcatgtcccaggcattctctctgtatgaagatgaaatcagcgattccaaagcacagcttgctgccatcaccttgatcattggcacatttgaaaggatgaagtgcttcagtgaagagaatcatgaacctctgaggactcagtgtgcccttgctgcatccaaacttctaaagaaacctgatcagggccgagctgtgagcacctgtgcacatctcttctggtctggcagaaacacggacaaaaatggggaggagcttcacggaggcaagagggtaatggagtgcctaaaaaaagctctaaaaatagcaaatcagtgcatggacccctctctacaagtgcagctattcatagaaattctgaacagatatatctacttctatgaaaaggaaaatgatgcggtaacaattcaggtattaaaccagcttatccaaaagattcgagaagacctcccgaatcttgaatccagtgaagaaacagagcagattaacaaacactttcataacacactggagcatttgcgcttgcggcgggaatcaccagaatccgaggggccaatttatgaaggactcatcctttaa

(19) United States
(12) Patent Application Publication
(10) Pub. No.: US 2023/0021959 A1
(43) Pub. Date: Jan. 26, 2023

(54) Stabilization of Retromer for the Treatment of Alzheimer's Disease and Other Neurodegenerative Disorders

(71) Applicants: The Trustees of Columbia University in the City of New York, New York, NY (US); Cornell University, Ithaca, NY (US); Meiragtx UK II Limited, London (GB)

(72) Inventors: Scott A. SMALL, New York, NY (US); Gregory PETSKO, New York, NY (US); Rebecca COX, New York, NY (US); Samuel D. WAKSAL, New York, NY (US); Alexandria FORBES, New York, NY (US); Yasir H. QURESHI, New York, NY (US)

(73) Assignees: The Trustees of Columbia University in the City of New York, New York, NY (US); Cornell University, Ithaca, NY (US); Meiragtx UK II Limited, London (GB)

(21) Appl. No.: 17/782,357

(22) PCT Filed: Dec. 7, 2020
(86) PCT No.: PCT/US2020/063627

§ 371 Date: Jun. 3, 2022

Related U.S. Application Data
(60) Provisional application No. 63/074,578, filed on Sep. 4, 2020.
(60) Provisional application No. 62/943,999, filed on Dec. 5, 2019.

(51) Int. Cl. C07K 14/47

(52) U.S. Cl. CPC C07K 14/47

(57) ABSTRACT

The present disclosure relates to methods and compositions for elevating and stabilizing retromer for treating and/or preventing Alzheimer's disease and other neurodegenerative disorders. Additionally, the disclosure relates to adenoviral based therapy for treating Alzheimer's disease (AD), and other neurodegenerative conditions such as Parkinson's Disease (PD), neuronal ceroid lipofuscinosis (NCL), and transmissible spongiform encephalopathies (TSEs or prion disease), multiple system atrophy (MSA), Down's syndrome, and hereditary spastic paraplegia, as well as tauopathies such as progressive supranuclear palsy (PSP), frontotemporal lobar dementia linked to chromosome 17q21-22 and its subtypes (FTLD-17/FTLD-Tau), Lewy Body Disease (LBD), amyotrophic lateral sclerosis (ALS), frontal-temporal degeneration (FTD), ALS-FTD, and chronic traumatic encephalopathy (CTE).

### CROSS REFERENCE TO RELATED APPLICATION

This application claims priority to U.S. Provisional Applications Nos. 62/943,999 filed on Dec. 5, 2019 and 63/074,578 filed on Sep. 4, 2020, both of which are incorporated by reference herein in their entirety.

### FIELD

The present disclosure relates to methods and compositions for elevating and stabilizing retromer for treating and/or preventing Alzheimer's disease and other neurodegenerative disorders.

### BACKGROUND

Alzheimer's disease (AD) has been characterized as a disease of misfolded proteins and neuroinflammation. AD therapeutics however aimed at amyloid, tau, cholinesterase inhibitors, anti-inflammatory compounds, and alternative therapies such as memantine and nutritional supplements have failed, and the disease remains a major source of mortality, morbidity and financial burden. Failure of AD clinical trials have forced researchers to investigate further the causality of the disease. Numerous preclinical studies have examined novel AD associated genes, intracellular protein homeostasis pathways, interaction of neurons with their microenvironment and with glial cells. Several investigations are still ongoing.

Recent genetic and cell biological findings in Alzheimer's disease have implicated ‘endosomal trafficking’ as playing central role in disease pathophysiology. Current literature advocates that four classes of genes are implicated in AD. These gene classes are: 1) endosomal trafficking; 2) cholesterol metabolism; 3) immune response; and 4) amyloid precursor protein (APP) processing. All four of these gene classes are linked to endosomal trafficking defects; directly or indirectly. Endosomal trafficking defects have also been implicated in other neurodegenerative diseases such as Parkinson's disease (PD), transmissible spongiform encephalopathies (TSEs or prion diseases), and Neuronal Ceroid Lipofuscinosis (NCL).

Retromer is a protein complex associated with endosomal organelles and controls trafficking of certain cellular cargo molecules within tubular vesicular carriers to the trans Golgi network. Defects in this trafficking have been linked to various neurodegenerative diseases (Small and Petsko 2015; Anderson et al. 2014). Neurodegeneration is the umbrella term for the progressive loss of structure or function of neurons, including death of neurons. Many neurodegenerative diseases including amyotrophic lateral sclerosis (ALS), Parkinson's Disease (PD), Alzheimer's Disease (AD), and Huntington's occur as a result of neurodegenerative processes.

The use of small molecules to improve retromer complex function has been described. However, developing a successful drug that will display positive pharmacological dynamics in vivo will be difficult and could take many years. There is an urgent need for effective treatments for these neurodegenerative diseases, and currently there are no gene-based therapies that would offer long-term benefits.

### SUMMARY

The present disclosure relates to compositions and methods that can be used to treat a subject (e.g., a mammalian subject, such as a human subject) that has, or is at risk of developing, a neurodegenerative disease, including but not limited to Alzheimer's disease (AD), Parkinson's disease (PD), neuronal ceroid lipofuscinosis (NCL), transmissible spongiform encephalopathies (TSEs or prion disease), Down's syndrome, hereditary spastic paraplegia (HSP), and multiple system atrophy (MSA), as well as tauopathies such as progressive supranuclear palsy (PSP), frontotemporal lobar dementia linked to chromosome 17q21-22 and its subtypes (FTLD-17/FTLD-Tau), Lewy Body Disease (LBD), amyotrophic lateral sclerosis (ALS), frontal-temporal degeneration (FTD), ALS-FTD, and chronic traumatic encephalopathy (CTE).

Using the compositions and methods of the disclosure, a subject (e.g., a mammalian subject, such as a human subject) that has or is at risk of developing a disease described above may be administered a composition containing a transgene encoding one or more of the retromer proteins described herein. The composition may comprise a vector, for example, a viral vector, such as an adeno-associated virus (AAV) vector. In some embodiments, the subject is administered a second composition containing a transgene encoding one or more of the retromer proteins described herein. The second composition may comprise a vector, for example, a viral vector, such as an AAV vector. In some embodiments, the subject is administered a third composition containing a transgene encoding one or more of the retromer proteins described herein. The third composition may comprise a vector, for example, a viral vector, such as an AAV vector.

In the first aspect, the disclosure features a composition containing a transgene encoding retromer core protein VPS35 and/or VPS26a and/or VPS26b. In embodiments, the transgene encodes VPS35. In embodiments, the transgene encodes VPS26a and/or VPS26b. In embodiments, the transgene encodes VPS35 and either VPS26a or VPS26b. In embodiments, the transgene encodes VPS35 and VPS26a. In embodiments, the transgene encodes VPS35 and VPS26b. In embodiments, the transgene encodes VPS26a and VPS26b. In other aspects the composition comprises two transgenes with one of the transgenes encoding VPS35 and the other encoding VPS26a or VPS26b. In other aspects, the composition comprises two transgenes with one of the transgenes encoding VPS26a and the other encoding VPS26b. In other aspects the composition comprises three transgenes with one of the transgenes encoding VPS35, another encoding VPS26a, and another encoding VPS26b.

In some embodiments, the transgene encodes retromer core protein VPS35. In some embodiments, the retromer core protein VPS35 protein is human. The retromer core protein VPS35 encoded by the transgene may have an amino acid sequence that is at least 85% identical to the amino acid sequence of VPS35 (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of VPS35). In some embodiments, the retromer core protein VPS35 encoded by the transgene has an amino acid sequence that is at least 90% identical to the amino acid sequence of VPS35 (e.g., an amino acid sequence that is 90%, 91%, 92%, 93%, 94%, 95%, 96%, 97%, 98%, 99%, or 100% identical to the amino acid sequence of VPS35). In some embodiments, the retromer core protein VPS35 encoded by the transgene has an amino acid sequence that is at least 95% identical to the amino acid sequence of VPS35 (e.g., an amino acid sequence that is 95%, 96%, 97%, 98%, 99%, or 100% identical to the amino acid sequence of VPS35). In some embodiments, the retromer core protein VPS35 encoded by the transgene has an amino acid sequence that differs from VPS35 by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the retromer core protein VPS35 encoded by the transgene has an amino acid sequence that differs from VPS35 by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions).

In some embodiments, the transgene encoding retromer core protein VPS35 comprises human VPS35 (Gene ID 55737). In some embodiments, the transgene encoding retromer core protein VPS35 has a nucleic acid sequence that is at least 70% identical to the nucleic acid sequence encoding VPS35 (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding VPS35). In some embodiments, the transgene encoding retromer core protein VPS35 has a nucleic acid sequence that is at least 85% identical to the nucleic acid sequence encoding VPS35 (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence encoding VPS35). In some embodiments, the transgene encoding retromer core protein VPS35 has a nucleic acid sequence that is at least 90% identical to the nucleic acid sequence encoding VPS35 (e.g., a nucleic acid sequence that is 90%, 91%, 92%, 93%, 94%, 95%, 96%, 97%, 98%, 99%, or 100% identical to the nucleic acid sequence encoding VPS35). In some embodiments, the transgene encoding retromer core protein VPS35 has a nucleic acid sequence that is at least 95% identical to the nucleic acid sequence encoding VPS35 (e.g., a nucleic acid sequence that is 95%, 96%, 97%, 98%, 99%, or 100% identical to the nucleic acid sequence encoding VPS35). In some embodiments, the transgene encoding retromer core protein VPS35 is codon optimized.

In some embodiments, the transgene encodes retromer core protein VPS26a. In some embodiments, the retromer core protein VPS26a protein is human. The retromer core protein VPS26a encoded by the transgene may have an amino acid sequence that is at least 85% identical to the amino acid sequence of VPS26a (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of VPS26a). In some embodiments, the retromer core protein VPS26a encoded by the transgene has an amino acid sequence that is at least 90% identical to the amino acid sequence of VPS26a (e.g., an amino acid sequence that is 90%, 91%, 92%, 93%, 94%, 95%, 96%, 97%, 98%, 99%, or 100% identical to the amino acid sequence of VPS26a). In some embodiments, the retromer core protein VPS26a encoded by the transgene has an amino acid sequence that is at least 95% identical to the amino acid sequence of VPS26a (e.g., an amino acid sequence that is 95%, 96%, 97%, 98%, 99%, or 100% identical to the amino acid sequence of VPS26a). In some embodiments, the retromer core protein VPS26a encoded by the transgene has an amino acid sequence that differs from VPS26a by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the retromer core protein VPS26a has an amino acid sequence that differs from VPS26a by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions).

In additional embodiments, the transgene encoding retromer core protein VPS26a comprises human VPS26a (Gene ID 9559). In some embodiments, the transgene encoding retromer core protein VPS26a has a nucleic acid sequence that is at least 70% identical to the nucleic acid sequence encoding VPS26a (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding VPS26a). In some embodiments, the transgene encoding retromer core protein VPS26a has a nucleic acid sequence that is at least 85% identical to the nucleic acid sequence encoding VPS26a (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of encoding VPS26a). In some embodiments, the transgene encoding retromer core protein VPS26a has a nucleic acid sequence that is at least 90% identical to the nucleic acid sequence encoding VPS26a (e.g., a nucleic acid sequence that is 90%, 91%, 92%, 93%, 94%, 95%, 96%, 97%, 98%, 99%, or 100% identical to the nucleic acid sequence of encoding VPS26a). In some embodiments, the transgene encoding retromer core protein VPS26a has a nucleic acid sequence that is at least 95% identical to the nucleic acid sequence encoding VPS26a (e.g., a nucleic acid sequence that is 95%, 96%, 97%, 98%, 99%, or 100% identical to the nucleic acid sequence of encoding VPS26a). In some embodiments, the transgene encoding retromer core protein VPS26a is codon optimized.

In some embodiments, the transgene encodes retromer core protein VPS26b. In some embodiments, the retromer core protein VPS26b protein is human. The retromer core protein VPS26b encoded by the transgene may have an amino acid sequence that is at least 85% identical to the amino acid sequence of VPS26b (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of VPS26b). In some embodiments, the retromer core protein VPS26b encoded by the transgene has an amino acid sequence that is at least 90% identical to the amino acid sequence of VPS26b (e.g., an amino acid sequence that is 90%, 91%, 92%, 93%, 94%, 95%, 96%, 97%, 98%, 99%, or 100% identical to the amino acid sequence of VPS26b). In some embodiments, the retromer core protein VPS26b encoded by the transgene has an amino acid sequence that is at least 95% identical to the amino acid sequence of VPS26b (e.g., an amino acid sequence that is 95%, 96%, 97%, 98%, 99%, or 100% identical to the amino acid sequence of VPS26b). In some embodiments, the retromer core protein VPS26b encoded by the transgene has an amino acid sequence that differs from VPS26b by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the retromer core protein VPS26b encoded by the transgene has an amino acid sequence that differs from VPS26b by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions).

In additional embodiments, the transgene encoding retromer core protein VPS26b comprises human VPS26b (Gene ID 112936). In some embodiments, the transgene encoding the retromer core protein VPS26b has a nucleic acid sequence that is at least 70% identical to the nucleic acid sequence encoding VPS26b (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence encoding VPS26b). In some embodiments, the transgene encoding the retromer core protein VPS26b has a nucleic acid sequence that is at least 85% identical to the nucleic acid sequence encoding VPS26b (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence encoding VPS26b). In some embodiments, the transgene encoding the retromer core protein VPS26b has a nucleic acid sequence that is at least 90% identical to the nucleic acid sequence encoding VPS26b (e.g., a nucleic acid sequence that is 90%, 91%, 92%, 93%, 94%, 95%, 96%, 97%, 98%, 99%, or 100% identical to the nucleic acid sequence of encoding VPS26b). In some embodiments, the transgene encoding the retromer core protein VPS26b has a nucleic acid sequence that is at least 95% identical to the nucleic acid sequence encoding VPS26b (e.g., a nucleic acid sequence that is 95%, 96%, 97%, 98%, 99%, or 100% identical to the nucleic acid sequence encoding VPS26b). In some embodiments, the transgene encoding the retromer VPS26b core protein is codon optimized.

In some embodiments of the preceding aspect, the composition comprises a vector, such as a viral vector. The viral vector may be, for example, an AAV vector, adenovirus vector, lentivirus vector, retrovirus vector, poxvirus vector, baculovirus vector, herpes simplex virus vector, vaccinia virus vector, or a synthetic virus vector (e.g., a chimeric virus, mosaic virus, or pseudotyped virus, and/or a virus that contains a foreign protein, synthetic polymer, nanoparticle, or small molecule).

In some embodiments of the preceding aspect, the viral vector is an AAV vector, such as an AAV1 (i.e., an AAV containing AAV1 inverted terminal repeats (ITRs) and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), or AAVrh.10 (i.e., an AAV containing AAVrh.10 ITRs and AAVrh.10 capsid proteins).

In some embodiments of the preceding aspect, the viral vector is a pseudotyped AAV vector, containing ITRs from one AAV serotype and capsid proteins from a different AAV serotype. In some embodiments, the pseudotyped AAV is AAV2/9 (i.e., an AAV containing AAV2 ITRs and AAV9 capsid proteins). In some embodiments, the pseudotyped AAV is AAV2/10 (i.e., an AAV containing AAV2 ITRs and AAV10 capsid proteins).

In some embodiments of the preceding aspect, the AAV vector contains a recombinant capsid protein, such as a capsid protein containing a chimera of one or more of capsid proteins from AAV1, AAV2, AAV3, AAV4, AAV5, AAV6, AAV7, AAV8, AAV9, AAV10, AAVrh74, AAVrh.8, or AAVrh.10. In embodiments, the capsid is a variant AAV capsid such as the AAV2 variant rAAV2-retro (SEQ ID NO:44 from WO 2017/218842, incorporated herein by reference).

In certain embodiments, the viral vector is AAV10. For example, the composition may comprise AAV10 comprising a nucleic acid sequence comprising a transgene encoding retromer core protein VPS35 and/or retromer core protein VPS26a and/or a retromer core protein VPS26b.

In certain embodiments, the viral vector is AAV9. For example, the composition may comprise AAV9 comprising a nucleic acid sequence comprising a transgene encoding retromer core protein VPS35 and/or retromer core protein VPS26a and/or a retromer core protein VPS26b.

In certain embodiments, the viral vector is AAV2/10. For example, the composition may comprise AAV2/10 comprising a nucleic acid sequence comprising a transgene encoding retromer core protein VPS35 and/or retromer core protein VPS26a and/or a retromer core protein VPS26b.

In certain embodiments, the viral vector is AAV2/9. For example, the composition may comprise AAV2/9 comprising a nucleic acid sequence comprising a transgene encoding retromer core protein VPS35 and/or retromer core protein VPS26a and/or a retromer core protein VPS26b.

In certain embodiments, the viral vector is an AAV vector and the transgene encodes VPS35 retromer core protein. For example, the composition may comprise a recombinant AAV (rAAV), such as AAV10, comprising a nucleic acid sequence comprising a transgene encoding a functional VPS35 retromer core protein. For example, the composition may comprise a recombinant AAV (rAAV), such as AAV9, comprising a nucleic acid sequence comprising a transgene encoding a functional VPS35 retromer core protein. For example, the composition may comprise a recombinant AAV (rAAV), such as AAV2/9 or AAV2/10, comprising a nucleic acid sequence comprising a transgene encoding a functional VPS35 retromer core protein.

In certain embodiments, the viral vector is an AAV vector and the transgene encodes retromer core protein VPS26a. For example, the composition may comprise a recombinant AAV (rAAV), such as AAV10, comprising a nucleic acid sequence comprising a transgene encoding a functional VPS26a retromer core protein. For example, the composition may comprise a recombinant AAV (rAAV), such as AAV9, comprising a nucleic acid sequence comprising a transgene encoding a functional retromer core protein VPS26a. For example, the composition may comprise a recombinant AAV (rAAV), such as AAV2/9 or AAV2/10, comprising a nucleic acid sequence comprising a transgene encoding a functional retromer core protein VPS26a.

In certain embodiments, the viral vector is an AAV vector and the transgene encodes retromer core protein VPS26b. For example, the composition may comprise a recombinant AAV (rAAV), such as AAV10, comprising a nucleic acid sequence comprising a transgene encoding a functional VPS26b retromer core protein. For example, the composition may comprise a recombinant AAV (rAAV), such as AAV9, comprising a nucleic acid sequence comprising a transgene encoding a functional retromer core protein VPS26b. For example, the composition may comprise a recombinant AAV (rAAV), such as AAV2/9 or AAV2/10, comprising a nucleic acid sequence comprising a transgene encoding a functional retromer core protein VPS26b.

In some embodiments of any of the above aspects of the disclosure, the composition comprises a liposome, vesicle, synthetic vesicle, exosome, synthetic exosome, dendrimer, or nanoparticle.

In some embodiments of any of the above aspects of the disclosure, the transgene is operably linked to a promoter that induces expression of the transgene in a neuron. The promoter may be, for example, a chicken beta actin promoter, cytomegalovirus (CMV) promoter, myosin light chain-2 promoter, alpha actin promoter, troponin 1 promoter, Na+/Ca2+ exchanger promoter, dystrophin promoter, creatine kinase promoter, alpha7 integrin promoter, brain natriuretic peptide promoter, alpha B-crystallin/small heat shock protein promoter, alpha myosin heavy chain promoter, or atrial natriuretic factor promoter.

In some embodiments of any of the above aspects of the disclosure, the transgene is operably linked to an enhancer that induces expression of the transgene in a neuron. Exemplary enhancers that may be used in conjunction with the compositions and methods of the disclosure are a CMV enhancer, a myocyte enhancer factor 2 (MEF2) enhancer, and a MyoD enhancer.

In another aspect, the disclosure features a method for treating a degenerative disease or disorder in a subject in need thereof by administering one or more compositions comprising one or more viral vectors according to the proceeding embodiments. In some embodiments, the composition is administered to the subject as soon as, or immediately after, the subject is diagnosed as having a degenerative disease or disorder. In embodiments, the degenerative disease or disorder is a neurodegenerative disease, such as Alzheimer's disease (AD), Parkinson's disease, neuronal ceroid lipofuscinosis (NCL), transmissible spongiform encephalopathies (TSEs or prion disease), multiple system atrophy (MSA), Down's syndrome, and hereditary spastic paraplegia (HSP), as well as tauopathies such as progressive supranuclear palsy (PSP), frontotemporal lobar dementia linked to chromosome 17q21-22 and its subtypes (FTLD-17/FTLD-Tau), Lewy Body Disease (LBD), amyotrophic lateral sclerosis (ALS), frontal-temporal degeneration (FTD), ALS-FTD, and chronic traumatic encephalopathy (CTE).

In another aspect, the disclosure features a method for treating a degenerative disease or disorder in a subject in need thereof by administering one or more compositions comprising a transgene encoding retromer core protein VPS35 and/or VPS26a and/or VPS26b, as described in the foregoing paragraphs. In embodiments, the composition comprises a transgene encoding VPS35 and a transgene encoding VPS26a. In embodiments, the composition comprises a transgene encoding VPS35 and a transgene encoding VPS26b. In embodiments, the composition comprises a transgene encoding VPS26a and a transgene encoding VPS26b. In embodiments, the composition comprises a transgene encoding VPS35, a transgene encoding VPS26a, and a transgene encoding VPS26b. In some embodiments, the composition is administered to the subject as soon as, or immediately after, the subject is diagnosed as having a degenerative disease or disorder. In embodiments, the degenerative disease or disorder is a neurodegenerative disease, such as Alzheimer's disease (AD), Parkinson's disease, neuronal ceroid lipofuscinosis (NCL), transmissible spongiform encephalopathies (TSEs or prion disease), Down's syndrome, hereditary spastic paraplegia (HSP), and multiple system atrophy (MSA), as well as tauopathies such as progressive supranuclear palsy (PSP), frontotemporal lobar dementia linked to chromosome 17q21-22 and its subtypes (FTLD-17/FTLD-Tau), Lewy Body Disease (LBD), amyotrophic lateral sclerosis (ALS), frontal-temporal degeneration (FTD), ALS-FTD, and chronic traumatic encephalopathy (CTE).

In some embodiments, the method includes administering to the subject a therapeutically effective amount of a first composition containing a transgene encoding retromer core protein VPS35 and/or VPS26a and/or VPS26b, as described in the foregoing paragraphs. In some embodiments, the method further includes administering to the subject a therapeutically effective amount of a second composition containing a transgene encoding retromer core protein VPS35 and/or VPS26a and/or VPS26b, as described in the foregoing paragraphs. In embodiments, the method comprises administering a first composition comprising a transgene encoding VPS35 and administering a second composition comprising a transgene encoding VPS26a or VPS26b. In embodiments, the method comprises administering a first composition comprising a transgene encoding VPS26a or VPS26b and administering a second composition comprising a transgene encoding VPS35. In embodiments, the method comprises administering a first composition comprising a transgene encoding VPS26a and administering a second compositions composition comprising a transgene encoding VPS26b. In embodiments, the method comprises administering a first composition comprising a transgene encoding VPS26b and administering a second compositions composition comprising a transgene encoding VPS26a.

In some embodiments, the method further includes administering to the subject a therapeutically effective amount of a third composition containing a transgene encoding retromer core protein VPS35 and/or VPS26a and/or VPS26b, as described in the foregoing paragraphs. In embodiments, the first, second and third compositions comprise a transgene encoding VPS35, VPS26a, or VPS26b, respectively.

In some embodiments, the first and second compositions are administered to the subject at the same time. In some embodiments, the first, second and third compositions are administered to the subject at the same time.

In some embodiments, the second composition is administered to the subject after administration of the first composition to the subject. The second composition may be administered to the subject, for example, within one or more days or weeks of administration of the first composition to the subject. In some embodiments, the second composition is administered to the subject at least one month after administration of the first composition to the subject. In some embodiments, administration of the first composition continues while the second composition is administered to the subject.

In some embodiments, the third composition is administered to the subject after administration of the first and second composition to the subject. The third composition may be administered to the subject, for example, within one or more days or weeks of administration of the first and second composition to the subject. In some embodiments, the third composition is administered to the subject at least one month after administration of the first and second composition to the subject. In some embodiments, administration of the first and second composition continues while the third composition is administered to the subject.

In some embodiments, the first composition is administered to the subject by way of intravenous, intrathecal, intradermal, transdermal, parenteral, intramuscular, intranasal, subcutaneous, percutaneous, intratracheal, intraperitoneal, intraarterial, intravascular, inhalation, perfusion, lavage, and/or oral administration.

In some embodiments, the second composition is administered to the subject by way of intravenous, intrathecal, intradermal, transdermal, parenteral, intramuscular, intranasal, subcutaneous, percutaneous, intratracheal, intraperitoneal, intraarterial, intravascular, inhalation, perfusion, lavage, and/or oral administration.

In some embodiments, the third composition is administered to the subject by way of intravenous, intrathecal, intradermal, transdermal, parenteral, intramuscular, intranasal, subcutaneous, percutaneous, intratracheal, intraperitoneal, intraarterial, intravascular, inhalation, perfusion, lavage, and/or oral administration.

In a further aspect, the disclosure features a method of treating, preventing, and/or curing a neurodegenerative disease or disorder in a subject in need thereof. The method includes administering to the subject a therapeutically effective amount of a composition or compositions containing a transgene encoding retromer core protein VPS35 and/or retromer core protein VPS26a and/or a retromer core protein VPS26b.

In an additional aspect, the disclosure features a method of alleviating one or more symptoms associated with a neurodegenerative disease or disorder in a subject in need thereof. The method includes administering to the subject a therapeutically effective amount of a composition or compositions containing a transgene encoding retromer core protein VPS35 and/or retromer core protein VPS26a and/or a retromer core protein VPS26b.

As part of the foregoing aspects, the disclosure also provides one or more compositions as described herein for use in a method as described herein. The disclosure also provides the use of one or more compositions as described herein for the manufacture of one or more medicaments for a method as described herein. The transgene may encode retromer core protein VPS35 and/or retromer core protein VPS26a and/or a retromer core protein VPS26b.

As part of the foregoing aspects, the disclosure therefore also provides a composition containing one or more transgenes encoding retromer core protein VPS35 and/or retromer core protein VPS26a and/or a retromer core protein VPS26b for use in treating, preventing, and/or curing a neurodegenerative disease or disorder. Furthermore provided is one or more compositions containing one or more transgenes encoding retromer core protein VPS35 and/or retromer core protein VPS26a and/or a retromer core protein VPS26b for use in alleviating one or more symptoms associated with a neurodegenerative disease or disorder.

As part of the foregoing aspects, the disclosure also provides the use of one or more compositions containing one or more transgenes encoding retromer core protein VPS35 and/or retromer core protein VPS26a and/or a retromer core protein VPS26b for the manufacture of one or more medicaments for treating, preventing, and/or curing a neurodegenerative disease or disorder. Furthermore provided is the use of one or more compositions containing one or more transgenes encoding retromer core protein VPS35 and/or retromer core protein VPS26a and/or a retromer core protein VPS26b for the manufacture of one or more medicaments for alleviating one or more symptoms associated with a neurodegenerative disease or disorder.

In some embodiments of any of the foregoing aspects, the disease or disorder is a neurodegenerative disease or disorder. In some embodiments of any of the foregoing aspects, the disease or disorder is Alzheimer's disease (AD). In some embodiments of any of the foregoing aspects, the disease or disorder is Parkinson's disease. In some embodiments of any of the foregoing aspects, the disease or disorder is neuronal ceroid lipofuscinosis (NCL). In some embodiments of any of the foregoing aspects, the disease or disorder is transmissible spongiform encephalopathies (TSEs or prion disease). In some embodiments of any of the foregoing aspects, the disease or disorder is multiple system atrophy (MSA). In some embodiments of any of the foregoing aspects, the disease or disorder is progressive supranuclear palsy (PSP). In some embodiments of any of the foregoing aspects, the disease or disorder is frontotemporal lobar dementia linked to chromosome 17q21-22 and its subtypes (FTLD-17/FTLD-Tau). In some embodiments of any of the foregoing aspects, the disease or disorder is chronic traumatic encephalopathy (CTE). In some embodiments of any of the foregoing aspects, the disease or disorder is Down's syndrome. In some embodiments of any of the foregoing aspects, the disease or disorder is HSP. In some embodiments of any of the foregoing aspects, the disease or disorder is LBD. In some embodiments of any of the foregoing aspects, the disease or disorder is ALS. In some embodiments of any of the foregoing aspects, the disease or disorder is FTD or ALS-FTD.

In another aspect, the disclosure features a kit containing the composition of the preceding aspect. The kit may further contain a package insert, such as a package insert instructing a user of the kit to administer the composition to a subject in accordance with the method of any of the above aspects or embodiments of the disclosure.

### BRIEF DESCRIPTION OF THE DRAWINGS

For the purpose of illustrating the invention, there are depicted in drawings certain embodiments of the invention. However, the invention is not limited to the precise arrangements and instrumentalities of the embodiments depicted in the drawings.

FIG. 1 shows a backbone rendering (PyMOL, Schroedinger, Inc.) of the three-dimensional structure of retromer's cargo recognition core highlighting the interaction of VPS35 (orange) with VPS29 (red) and VPS26a (green). Vps26b binds to Vps35 in a virtually identical fashion (Collins et al. 2008; Shi et al. 2006). Atomic coordinates (PDB file 6vac) taken from the cryoEM structure of the mouse heterotrimer (Kendall et al. 2020).

FIG. 2 shows VPS35 expression alone is insufficient to elevate retromer's trimer and function. FIG. 2A are representative immunoblots showing retromer and Sorll expression levels following AAV9-VPS35-HA. AAV9-GFP and AAV9-EV (empty vector) were used as controls. FIG. 2B is a bar graph showing mean levels of retromer components and Sorll, normalized to actin; Control (left hand bars, dark grey, n=23), VPS35 Overexpression (right hand bars, red, n=16). ***P<0.001, ns=not significant.

FIG. 3 shows the maps of the plasmids used in the Examples. FIG. 3A shows the empty chassis control plasmid map. FIG. 3B shows the GFP control plasmid map. FIG. 3C shows the VPS35 plasmid map. FIG. 3D shows the VPS26a plasmid map. FIG. 3E shows VPS26b plasmid map.

FIG. 4 shows the optimization of the combined VPS35 and VPS26 expression in neuroblastoma cells. FIG. 4A are representative immunoblots showing retromer expression levels following transfection with plasmids containing VPS35, VPS26a, and VPS26b alone or dual transfection of VPS35 along with VPS26a or VPS26b. GFP or empty backbone plasmids were used as controls. To properly control for the amount of plasmid DNA/lipofectamine complexes introduced in each condition, a control plasmid (GFP or empty backbone) was included whenever only one component of retromer was transfected. VPS29 shows two distinct bands, which represent two different isoforms of this protein in these N2a cells. FIGS. 4B through 4E are graphs of the mean levels of retromer core protein (VPS35, VPS26a, VPS26b, VPS29) in neuroblastoma cells, normalized to actin, transfected with viral vectors containing empty vector (EV) and GFP as a control (EV+GFP), VPS35 alone, VPS26a alone, VPS26b alone, VPS35 vector+VPS26a vector, and VPS35 vector+VPS26b vector. FIG. 4B shows VPS35. FIG. 4C shows VPS26a. FIG. 4D shows VPS26b. FIG. 4E shows VPS29. Control (dark grey, n=15), VPS35 (n=30), VPS26a (n=30), and VPS26b (n=15) overexpression alone (blue), VPS35+VPS26 combinatorials (red, n=15). ***P<0.001, **P<0.01, !P=0.07, ns=not significant.

FIG. 5 shows that combined VPS35 and VPS26 expression synergizes retromer function in neurons. FIG. 5A are representative immunoblots showing retromer expression levels following transduction with AAV9 vectors containing VPS35, VPS26a, and VPS26b. AAV9-GFP and AAV9-EV (AAV9 containing empty backbone plasmid) were used as controls. The experimental AAV9 vectors were expressed in neurons in all possible combinations: Single protein expression (VPS35 alone, VPS26a alone, VPS26b alone); double protein expression (VPS35+VPS26a, VPS35+VPS26b, VPS26b+VPS26a); and triple protein expression (VPS35+VPS26a+VPS26b). To properly control for the amount of DNA and AAV9 introduced in each condition, a control AAV9 (GFP or EV) was included whenever only one component of retromer was transduced. FIGS. 5B through 5E are bar graphs of the mean levels of retromer core protein (VPS35, VPS26a, VPS26b) in neuroblastoma cells, normalized to actin, transfected with one or more of five viral vectors (AAV9) containing empty vector (EV), GFP, VPS35, VPS26a, or VPS26b. Results are shown for empty vector (EV)+GFP as a control, VPS35 vector alone, VPS26a vector alone, VPS26b vector alone, VPS35 vector+VPS26a vector, VPS35 vector+VPS26b vector, VPS35 vector+VPS26a vector+VPS26b vector, and VPS26a vector+VPS26b vector. FIG. 5B shows VPS35. FIG. 5C shows VPS29. FIG. 5D shows VPS26a. FIG. 5E shows VPS26b. Control (dark grey, n=9), single overexpression of VPS35, VPS26a, or VPS26b (blue, n=18), VPS35+VPS26a or VPS26b combinatorials (red, n=9), VPS35+VPS26a+VPS26b (dark red, n=9), VPS26a+VPS26b (pink, n=9). ***P<0.001, **P<0.01, *P<0.05, ns=not significant.

FIG. 6 shows that the combined VPS35 and VPS26 expression synergizes retromer function in neurons. FIG. 6A are representative immunoblots showing Sorll expression levels following transduction with AAV9 vectors containing VPS35, VPS26a, and VPS26b. AAV9-GFP and AAV9-EV (AAV9 containing empty backbone plasmid) were used as controls. The experimental AAV9 vectors were expressed in neurons in all possible combinations: Single protein expression (VPS35 alone, VPS26a alone, VPS26b alone); double protein expression (VPS35+VPS26a, VPS35+VPS26b, VPS26b+VPS26a); and triple protein expression (VPS35+VPS26a+VPS26b). To properly control for the amount of DNA and AAV9, introduced in each condition, a control AAV9 (GFP or EV) was included whenever only one component of retromer was transduced. FIG. 6B is a bar graph of the levels of Sorll in neurons, normalized to actin, transfected with one or more of five viral vectors (AAV9) containing empty vector (EV), GFP, VPS35, VPS26a, or VPS26b. Results are shown for empty vector (EV)+GFP as a control, VPS35 vector alone, VPS26a vector alone, VPS26b vector alone, VPS35 vector+VPS26a vector, VPS35 vector+VPS26b vector, VPS35 vector+VPS26a vector+VPS26b vector, and VPS26a vector+VPS26b vector. Control (dark grey, n=9), single overexpression of VPS35, VPS26a, or VPS26b (blue, n=18), VPS35+VPS26a or VPS26b combinatorials (red, n=9), VPS35+VPS26a +VPS26b (dark red, n=9), VPS26a+VPS26b (pink, n=9). ***P<0.001, ns=not significant. FIGS. 6C and 6D are scatter plots generated from a multivariate regression, demonstrating that VPS26a (t=5.6, p=1.4E-7) and VPS26b (F=7.2, p=3.2E-11) independently correlate with Sorll levels. FIG. 6C shows VPS26a. FIG. 6D shows VPS26b.

### DETAILED DESCRIPTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “subject” as used in this application refers to animals in need of therapeutic or prophylactic treatment. Subjects include mammals, such as canines, felines, rodents, bovine, equines, porcines, ovines, and primates. Thus, the compositions and methods can be used in veterinary medicine, e.g., to treat companion animals, farm animals, laboratory animals in zoological parks, and animals in the wild. The compositions and methods disclosed herein are particularly desirable for human medical applications.

The term “patient” as used in this application means a human subject. In some embodiments, the “patient” is known or suspected of having a neurodegenerative disease or disorder including but not limited to Alzheimer's disease (AD), Parkinson's disease, neuronal ceroid lipofuscinosis (NCL), transmissible spongiform encephalopathies (TSEs or prion disease), multiple system atrophy, Down's syndrome, and hereditary spastic paraplegia (HSP), (MSA), as well as tauopathies such as progressive supranuclear palsy (PSP), frontotemporal lobar dementia linked to chromosome 17q21-22 and its subtypes (FTLD-17/FTLD-Tau), Lewy Body Disease (LBD), amyotrophic lateral sclerosis (ALS), frontal-temporal degeneration (FTD), ALS-FTD, and chronic traumatic encephalopathy (CTE). In some embodiments, the “patient” is known or suspected of having a disorder or disease that is associated with endosomal trafficking, e.g., retromer dysfunction.

The phrase “therapeutically effective amount” is used herein to mean an amount sufficient to cause an improvement in a clinically significant condition in the subject, or delays or minimizes or mitigates one or more symptoms associated with the disease or disorder, or results in a desired beneficial change of physiology in the subject.

The terms “treat”, “treatment”, and the like refer to a means to slow down, relieve, ameliorate or alleviate at least one of the symptoms of the disease or disorder, or reverse the disease or disorder after its onset.

The terms “prevent”, “prevention”, and the like refer to acting prior to overt disease or disorder onset, to prevent the disease or disorder from developing or minimize the extent of the disease or disorder, or slow its course of development.

The term “cure” and the like means to heal, to make well, or to restore to good health or to allow a time without recurrence of disease so that the risk of recurrence is small.

The term “in need thereof” would be a subject known or suspected of having or being at risk of having a neurodegenerative disease or disorder including but not limited to Alzheimer's disease (AD), Parkinson's disease, neuronal ceroid lipofuscinosis (NCL), transmissible spongiform encephalopathies (TSEs or prion disease), multiple system atrophy, Down's syndrome, and hereditary spastic paraplegia (HSP), (MSA), as well as tauopathies such as progressive supranuclear palsy (PSP), frontotemporal lobar dementia linked to chromosome 17q21-22 and its subtypes (FTLD-17/FTLD-Tau), Lewy Body Disease (LBD), amyotrophic lateral sclerosis (ALS), frontal-temporal degeneration (FTD), ALS-FTD, and chronic traumatic encephalopathy (CTE).

The term “agent” as used herein means a substance that produces or is capable of producing an effect and would include, but is not limited to, vectors, chemicals, pharmaceuticals, biologics, small organic molecules, antibodies, nucleic acids, peptides, and proteins.

As used herein, the term “carrier” refers to a diluent, adjuvant, excipient, or vehicle with which the therapeutic is administered, and includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art.

The term “pharmaceutically acceptable” refers to molecular entities and compositions that do not produce an allergic or similar untoward reaction when administered to a host, such as gastric upset, dizziness and the like, when administered to a human, and approved by a regulatory agency of the Federal or a state government or listed in the U.S. Pharmacopeia or other generally recognized pharmacopeia for use in animals, and more particularly in humans.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

In some aspects, the disclosure provides isolated adeno-associated viral vectors (AAVs). As used herein with respect to AAVs, the term “isolated” refers to an AAV that has been isolated from its natural environment (e.g., from a host cell, tissue, or subject) or artificially produced. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities.

Methods for obtaining recombinant AAVs having a desired capsid protein have been described (see, for example, U.S. Pat. No. 7,906,111). A number of different AAV capsid proteins have been described, for example, those disclosed in Gao, et al., J. Virology 78(12):6381-6388 (June 2004); Gao, et al., Proc Natl Acad Sci USA 100(10):6081-6086 (May 13, 2003); and U.S. Pat. Nos. 7,906,111; 8,999,678. In embodiments for the desired packaging of the presently described constructs and methods, the recombinant AAV may be AAV9 or AAV10 vector and capsid. However, it is noted that other suitable AAVs such as rAAVrh.8 and rAAVrh.10, or other similar vectors may be adapted for use in the present methods and compositions. Typically, the methods involve culturing a host cell which contains a nucleic acid sequence encoding an AAV capsid protein or fragment thereof; a functional rep gene; a recombinant AAV vector composed of AAV inverted terminal repeats (ITRs) and a transgene; and sufficient helper functions to permit packaging of the recombinant AAV vector into the AAV capsid proteins.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters.

The recombinant AAV vector, rep sequences, cap sequences, and helper functions for producing the rAAV may be delivered to the packaging host cell using any appropriate genetic element (vector). The selected genetic element may be delivered by any suitable method, including those described herein. See, e.g., Fisher et al, J. Virology 70:520-532 (1993) and U.S. Pat. No. 5,478,745.

In some embodiments, recombinant AAVs may be produced using the triple transfection method (e.g., as described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

As used herein, the terms “AAV1,” “AAV2,” “AAV3,” “AAV4,” and the like refer to AAV vectors containing ITRs from AAV1, AAV2, AAV3, or AAV4, respectively, as well as capsid proteins from AAV1, AAV2, AAV3, or AAV4, respectively. The terms “AAV2/1,” “AAV2/8,” “AAV2/9,” and the like refer to pseudotyped AAV vectors containing ITRs from AAV2 and capsid proteins from AAV1, AAV8, or AAV9, respectively.

With respect to transfected host cells, the term “transfection” is used to refer to the uptake of foreign DNA by a cell, and a cell has been “transfected” when exogenous DNA has been introduced inside the cell membrane. A number of transfection techniques are generally known in the art. See, e.g., Graham et al., Virology 52:456 (1973), Sambrook et al., Molecular Cloning, a Laboratory Manual, Cold Spring Harbor Laboratories, New York (1989), Davis et al., Basic Methods in Molecular Biology, Elsevier (1986), and Chu et al., Gene 13:197 (1981). Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAV vectors. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

With respect to cells, the term “isolated” refers to a cell that has been isolated from its natural environment (e.g., from a tissue or subject). The term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

The term “vector” includes any genetic element, such as a plasmid, phage, transposon, cosmid, chromosome, artificial chromosome, virus, or virion, which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “operatively linked,” “under control,” or “under transcriptional control” means that the promoter is in the correct location and orientation in relation to the nucleic acid to control RNA polymerase initiation and expression of the gene.

The term “expression vector” or “expression construct” or “construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA from a transcribed gene.

Standard methods in molecular biology are described Sambrook, Fritsch and Maniatis Molecular Cloning, A Laboratory Manual, Cold Spring Harbor Laboratory Press, Cold Spring Harbor, N.Y. (1982 & 1989 2nd Edition, 2001 3rd Edition); Sambrook and Russell Molecular Cloning, 3rd ed., Cold Spring Harbor Laboratory Press, Cold Spring Harbor, N.Y. (2001); Wu Recombinant DNA, Vol. 217, Academic Press, San Diego, Calif.) (1993). Standard methods also appear in Ausbel, et al. Current Protocols in Molecular Biology, Vols. 1-4, John Wiley and Sons, Inc. New York, N.Y. (2001).

### Retromers and Neurodegenerative Diseases

The genetics, cytopathology, and cell biology of Alzheimer's disease (AD) have converged on endosomal trafficking as a key defect in the pathogenesis of AD. Three lines of evidence have implicated retromer dysfunction in AD. First and foremost, genetic and gene expression studies have identified a growing number of retromer-related molecules associated with AD, including bona fide loss-of-function mutations. Second, retromer dysfunction recapitulates AD's cytopathology, characterized by enlarged and dysfunctional endosomes in which fragments of the amyloid precursor protein (APP) accumulate. Third, retromer dysfunction mistraffics a number of AD-related molecules, including APP in neurons and phagocytic receptors in microglia.

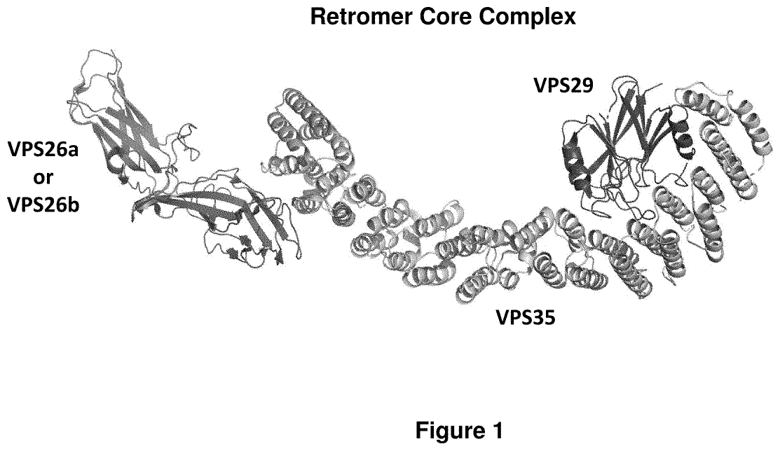

Retromer is a multiprotein complex that is a ‘master conductor’ of endosomal trafficking. Retromer's core is a trimer of three different proteins, making it technically a heterotrimer. The proteins are all members of the ‘Vacuolar Protein Sorting’ (VPS) family of proteins. VPS35 is the trimer core's central protein to which VPS29 and VPS26 bind. VPS26 is the only core protein that has two paralogs called VPS26a and VPS26b. Thus, neurons have two distinct retromer cores: VPS29-VPS35-VPS26a and VPS29-VPS35-VPS26b. See FIG. 1.

These core proteins when overexpressed, bind to the endogenous retromer components and can lead to increased retromer function. These proteins are very tightly autoregulated inside cells. To overcome this barrier two retromer proteins are co-expressed at the same time, as described herein.

Increasing VPS35 levels, either by pharmacological chaperones or via viral vectors, increases retromer's function. Compared to either VPS35 or VPS26, VPS29 protein may exist in a surplus. As shown herein, the co-expression of both VPS35 and VPS26a or both VPS35 and VPS26b has a synergistic effect on cellular levels of VPS35 and VPS26a or VPS35 and VPS26b, respectively.

Evidence for retromer function is also shown by an increase in Sorll levels by approximately 34%, an effect size that mirrors the degree of Sorll deficiency observed in Alzheimer's disease (Sager et al. 2007; Scherzer et al. 204; Dodson et al. 2006). A broader comparison on the effect of Sorll across the studies informs on retromer functionality. In the first neuronal study, when VPS35 was overexpressed alone, two out of the three retromer core proteins were robustly elevated, but with no effect on retromer function. In the second neuronal study, when thanks to synergism all three of the trimeric proteins were robustly elevated, this led to an increase in retromer function. This result provides primary empirical evidence that all three retromer core proteins need to be co-elevated to upregulate retromer's overall function. The studies herein show that by exploiting retromer's stoichiometry and protein-protein interactions there is no need to exogenously express all three proteins. Exogenously expressing VPS35 and VPS26 is sufficient to also upregulate the level of VPS29 and increase retromer's endosomal cargo recycling function.

It is also shown herein, importantly, that the levels of VPS26a and VPS26b are independent of one another, and therefore appropriate choice of combinatorials allows selective increase of one retromer heterotrimer over the other.

Described herein is a biologically based method to increase retromer levels and function in vivo: the overexpression of retromer by the use of recombinant AAV (adeno-associated adenovirus) technology. Establishing novel retromer-AAV tools to be used for retromer based therapeutics, will have a high impact, as this viral delivery system—recently approved for clinical applications—can bypass the obstacles that a small molecule would encounter within the organism (i.e., low absorption rates, degradation, toxicity, lack of target/organ specificity, blood brain barrier permeability). The compositions comprising AAV vectors and the retromer transgene(s) have many advantages including increased expression of the therapeutic agent, bypassing of strict protein autoregulation, the potential for long term expression of stabilized proteins, and increasing the half-life of the stabilized proteins.

Methods of Treating, Preventing, and/or Curing Neurodegenerative Diseases

Patients who would benefit from the administration of the described gene therapy include those diagnosed with a neurogenerative disease or disorder where endosomal trafficking defects are implicated including but not limited to Alzheimer's disease (AD), Parkinson's disease, neuronal ceroid lipofuscinosis (NCL), transmissible spongiform encephalopathies (TSEs or prion disease), multiple system atrophy (MSA), Down's syndrome and hereditary spastic paraplegia (HSP), as well as tauopathies such as progressive supranuclear palsy (PSP), frontotemporal lobar dementia linked to chromosome 17q21-22 and its subtypes (FTLD-17/FTLD-Tau), Lewy Body Disease (LBD), amyotrophic lateral sclerosis (ALS), frontal-temporal degeneration (FTD), ALS-FTD, and chronic traumatic encephalopathy (CTE).

In these patients, compositions containing a nucleic acid encoding one or more of the retromer core proteins (e.g., viral vectors, such as AAV vectors, containing such nucleic acids) may be administered to the patient. These compositions may be administered alone or in combination with other agents for the treatment of neurodegenerative diseases or disorders.

In some embodiments, the present disclosure provides methods of treating, preventing, curing, and/or reducing the severity or extent of a neurodegenerative disease or disorder, by administering to a subject in need thereof a therapeutically effective amount of a composition, or compositions, such as a viral vector (e.g., an AAV), comprising a nucleic acid encoding retromer core protein VPS35 and/or retromer core protein VPS26 and/or retromer core protein VPS26b. In some embodiments, the viral vector is an AAV, such as rAAV2-retro, AAV10, AAV2/10, AAV9 or an AAV2/9. In some embodiments, the composition or compositions (e.g., viral vector, such as an AAV) comprising a nucleic acid encoding retromer core protein VPS35 and/or retromer core protein VPS26a and/or retromer core protein VPS26b is administered as soon as neurodegenerative disease or disorder is diagnosed or suspected. In embodiments, the administered composition comprises a nucleic acid encoding VPS35 and either VPS26a or VPS26b. In embodiments, the method comprises administering one or more compositions comprising a nucleic acid encoding VPS35 and a nucleic acid encoding either VPS26a or VPS26b; ether simultaneously or sequentially. In embodiments, the method comprises administering one or more compositions comprising a nucleic acid encoding VPS35, a nucleic acid encoding VPS26a, and a nucleic acid encoding VPS26b; ether simultaneously or sequentially. In embodiments, the method comprises administering one or more compositions comprising a nucleic acid encoding VPS26a and a nucleic acid encoding VPS26b; ether simultaneously or sequentially.

In some embodiments, the amount of AAV vector comprising the transgene administered is about 4.2×10¹¹ or 4.2×10¹⁰ genome or vector or vector copies.

The disclosure also provides methods of treating, preventing, curing, and/or reducing the severity or extent of neurodegenerative disease or disorder by administering to a subject in need thereof a therapeutically effective amount of a first composition (e.g., viral vector, such as AAV) containing a nucleic acid encoding retromer core protein VPS35 and/or retromer core protein VPS26a and/or retromer core protein VPS26b and further comprising administering to the subject a therapeutically effective amount of a second composition (e.g., viral vector, such as AAV) containing a nucleic acid encoding retromer core protein VPS35 and/or retromer core protein VPS26a and/or retromer core protein VPS26b. In embodiments, the method comprises administering a therapeutically effective amount of a first composition containing a nucleic acid encoding retromer core protein VPS35 and a therapeutically effective amount of a second composition containing a nucleic acid encoding a retromer core protein VPS26a or VPS26b. In embodiments, the method comprises administering a therapeutically effective amount of a first composition containing a nucleic acid encoding retromer core protein VPS26a or VPS26b and a therapeutically effective amount of a second composition containing a nucleic acid encoding a retromer core protein VPS35.

The disclosure also provides methods of treating, preventing, curing, and/or reducing the severity or extent of neurodegenerative disease or disorder by administering to a subject in need thereof a therapeutically effective amount of a first composition (e.g., viral vector, such as AAV) containing a nucleic acid encoding retromer core protein VPS35 and/or retromer core protein VPS26a and/or retromer core protein VPS26b and further comprising administering to the subject a therapeutically effective amount of a second composition (e.g., viral vector, such as AAV) containing a nucleic acid encoding retromer core protein VPS35 and/or retromer core protein VPS26a and/or retromer core protein VPS26b and further comprising administering to the subject a therapeutically effective amount of a third composition (e.g., viral vector, such as AAV) containing a nucleic acid encoding retromer core protein VPS35 and/or retromer core protein VPS26a and/or retromer core protein VPS26b. In embodiments, the first, second and third compositions contain a nucleic acid encoding a retromer core protein VPS35, VPS26a or VPS26b, respectively.

In some embodiments, the first and second and third AAV vectors are each independently an AAV9 vector encoding retromer core protein VPS35 and/or retromer core protein VPS26a and/or VPS26b retromer core protein. In some embodiments, the first AAV vector and the second AAV vector and the third AAV vector are administered at the same time. In some embodiments, the first AAV vector is administered prior to the second AAV vector. In some embodiments, the second AAV vector is administered prior to the third AAV vector.

In some embodiments, the first composition (e.g., AAV vector) is administered as soon as neurodegenerative disease or disorder is diagnosed or suspected, and the second composition (e.g., AAV vector) is administered at a time point after the first composition. In some embodiments, the second composition (e.g., AAV vector) is administered within hours of the first composition (e.g., AAV vector). In some embodiments, the second composition (e.g., AAV vector) is administered within days of the first composition (e.g., AAV vector). In some embodiments, the second composition (e.g., AAV vector) is administered weeks after the first composition (e.g., AAV vector). In some embodiments, the first composition (e.g., AAV vector) and the second composition (e.g., AAV vector) are administered simultaneously at any given time point.

In some embodiments, the third composition (e.g., AAV vector) is administered at a time point after the second composition. In some embodiments, the third composition (e.g., AAV vector) is administered within hours of the second composition (e.g., AAV vector). In some embodiments, the third composition (e.g., AAV vector) is administered within days of the second composition (e.g., AAV vector). In some embodiments, the third composition (e.g., AAV vector) is administered weeks after the second composition (e.g., AAV vector).

In some embodiments, the first composition (e.g., AAV vector) and the second composition (e.g., AAV vector) and the third composition (e.g., AAV vector) are administered simultaneously at any given time point, including a time point when, or after, the neurodegenerative disease or disorder is diagnosed or suspected. In some embodiments, the three compositions (e.g., AAV vectors) are present within the same larger composition, and in some embodiments, the three are separate compositions.

In embodiments of the methods described herein, one or more compositions comprising one or more nucleic acids encoding VPS35 and VPS26b (which is preferentially expressed in the cortex) are administered to a subject that has, or is at risk of developing, a disorder in which endosomal trafficking defects occur primarily in the cortex and in which VPS35 is unaffected. Examples of cortical endosomal trafficking disorders in which VPS35 is unaffected include biomarker-negative sporadic AD, AD patients with SORL1 mutations, FTD, prion disease, and Down's syndrome.

In other embodiments of the methods described herein, one or more compositions comprising one or more nucleic acids encoding VPS35 and VPS26a (which is preferentially expressed in the subcortex) are administered to a subject that has, or is at risk of developing, a disorder in which endosomal trafficking defects occur primarily in subcortical regions, such as biomarker-negative sporadic PD, HSP, prion disease, and NCL.

In other embodiments of the methods described herein, one or more compositions comprising one or more nucleic acids encoding VPS35, VPS26a, and VPS26b are administered to a subject that has, or is at risk of developing, an endosomal trafficking neurological disorder in which the disease is more diffuse, such as Lewy Body Disease (LBD), prion disease, and ALS-FTD.

In addition to treating, preventing, curing, and/or reducing the severity or extent of neurodegenerative disease or disorder, in embodiments, the methods and compositions described herein are used to treat, prevent, cure and/or reduce the severity of other disorders or diseases that are associated with endosomal trafficking and retromer dysfunction.

### Recombinant AAV Vectors

“Recombinant AAV (rAAV) vectors” described herein generally include a transgene (e.g., encoding retromer core protein VPS35 and/or retromer core protein VPS26a and/or retromer core protein VPS26b). The transgene is flanked by 5′- and 3′-ITRs and may be operably linked to one or more regulatory elements in a manner that permits transgene transcription, translation, and/or expression in a cell of a target tissue. Such regulatory elements may include a promoter or enhancer, such as the chicken beta actin promoter or cytomegalovirus enhancer, among others described herein. The recombinant AAV genome is generally encapsidated by capsid proteins (e.g., from the same AAV serotype as that from which the ITRs are derived or from a different AAV serotype from that which the ITRs are derived). The AAV vector may then be delivered to a selected target cell type or tissue. In some embodiments, the transgene is a nucleic acid sequence, heterologous to the vector sequences, which encodes one or more of VPS35, VPS26a and/or VPS26b. Components of exemplary AAV vectors that may be used in conjunction with the compositions and methods of the disclosure are described herein.

Any AAV serotype or combination of AAV serotype can be used in the methods and compositions of the present disclosure. Because the methods and compositions of the present disclosure are for the treatment and cure of neurodegenerative diseases or disorders, AAV serotypes that target at least the central nervous system can be used in some embodiments and include but are not limited to AAV1, AAV2, AAV4, AAV5, AAV6, AAV7, AAV8, AAV9, and AAV10.

In some embodiments, AAV9 serotype, which has a wide tropism, is used. In some embodiments, an AAV2/9 is used.

### Components of AAV Vectors

The AAV vectors described herein may contain cis-acting 5′ and 3′ ITRs (See, e.g., Carter, in “Handbook of Parvoviruses”, ed., P. Tijsser, CRC Press, pp. 155 168 (1990)). The ITR sequences are typically about 145 bp in length. Preferably, substantially the entire sequences encoding the ITRs are used in the molecule, although some degree of minor modification of these sequences is permissible. (See, e.g., texts such as Sambrook et al, (1989) and Fisher et al., (1996)). An example of such a molecule is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types.

In addition to the elements identified above for recombinant AAV vectors, the vector may also include conventional control elements which are operably linked to the transgene in a manner which permits its transcription, translation and/or expression in a cell transfected with the plasmid vector or infected with the virus. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins. In some embodiments, operably linked coding sequences yield a fusion protein. In some embodiments, operably linked coding sequences yield a functional RNA (e.g., shRNA, miRNA). In some embodiments, operably linked coding sequences yield two or more separate functional proteins (e.g., VPS35 and VPS26a or VPS26b).

For nucleic acids encoding proteins, a polyadenylation sequence generally is inserted following the transgene sequences and before the 3′ AAV ITR sequence. An rAAV construct of the present disclosure may also contain an intron, desirably located between the promoter/enhancer sequence and the transgene. One possible intron sequence is derived from SV-40 and is referred to as the SV-40 T intron sequence.

Another vector element that may be used is an internal ribosome entry site (IRES). An IRES sequence is used to produce more than one polypeptide or protein from a single transcript. An IRES element may be used, for example, to express VPS35 and VPS26a, VPS35 and VPS26b, or VPS26a and VPS26b from the same AAV vector.

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors may optionally include 5′ leader or signal sequences.

Examples of constitutive promoters include, without limitation, a chicken beta actin promoter, a retroviral Rous sarcoma virus (RSV) LTR promoter (optionally with a RSV enhancer), a cytomegalovirus (CMV) promoter (optionally with a CMV enhancer), a SV40 promoter, a dihydrofolate reductase promoter, a β-actin promoter, a phosphoglycerol kinase (PGK) promoter, and a human elongation factor-la (EFla) promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Examples of inducible promoters regulated by exogenously supplied promoters include a zinc-inducible sheep metallothionine (MT) promoter, a dexamethasone (Dex)-inducible mouse mammary tumor virus (MMTV) promoter, a T7 polymerase promoter system (WO 98/10088); a ecdysone insect promoter (No et al., Proc. Natl. Acad. Sci. USA 93:3346-3351 (1996)), a tetracycline-repressible system (Gossen et al., Proc. Natl. Acad. Sci. USA 89:5547-5551 (1992)), a tetracycline-inducible system (Gossen et al., Science 268:1766-1769 (1995), a RU486-inducible system (Wang et al., Nat. Biotech. 15:239-243 (1997) and Wang et al., Gene Ther. 4:432-441 (1997)) and a rapamycin-inducible system (Magari et al., J. Clin. Invest. 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, a native promoter, or fragment thereof, for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner.

In some embodiments, one or more bindings sites for one or more of miRNAs are incorporated in a transgene of a rAAV vector, to inhibit the expression of the transgene in one or more tissues of a subject harboring the transgenes. The miRNA target sites in the mRNA may be in the 5′-UTR, the 3′-UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISCs and provide highly efficient inhibition of expression. The target site sequence may comprise a total of 5-100, 10-60, or more nucleotides. The target site sequence may comprise at least 5 nucleotides of the sequence of a target gene binding site.

For example, a 3′-UTR site which would inhibit the expression of the transgene in the liver can be incorporated into a transgene. This would be beneficial for transgenes which encode therapeutic proteins which are toxic to the liver as most of the virus administered (approximately 60 to 90%) is eventually found in the liver. Thus suppressing the therapeutic gene expression in liver relieves the burden from liver cells.

In some embodiments, the AAV vector will be modified to be a self-complementing AAV. A self-complementing AAV carries complementary sequence of the transgene (i.e., a double copy of the transgene). Self-complementation makes the gene more stable after it enters the cell.

### Transgene Coding Sequences

Nucleic acid sequences of transgenes described herein may be designed based on the knowledge of the specific composition (e.g., viral vector) that will express the transgene. For example, one type of transgene sequence includes a reporter sequence, which upon expression produces a detectable signal. In another example, the transgene encodes a therapeutic protein or therapeutic functional RNA. In another example, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. Appropriate transgene coding sequences will be apparent to the skilled artisan.

In embodiments, the transgenes encode a functional protein including but not limited to retromer core protein VPS35 and/or retromer core protein VPS26a and/or retromer core protein VPS26b. In embodiments, the transgene encodes VPS35 and either VPS26a or VPS26b. In embodiments, the transgene encodes VPS35, VPS26a and VPS26b. In embodiments, the transgene encodes VPS26a and VPS26b. In embodiments, the transgene encodes just one of VPS35, VPS26a, and VPS26b.

The amino acid sequence information can be obtained from the National Center for Biotechnology Information (NCBI) and are set forth below.

The gene encoding the human retromer core protein VPS35 (Gene ID: 55737) can be used to obtain a transgene encoding a functional retromer core protein VPS35 (SEQ ID NO: 1):

MPTTQQSPQDEQEKLLDEAIQAVKVQSFQMKRCLDKNKLMDALKHASNML
GELRTSMLSPKSYYELYMAISDELHYLEVYLTDEFAKGRKVADLYELVQY
AGNIIPRLYLLITVGVVYVKSFPQSRKDILKDLVEMCRGVQHPLRGLFLR
NYLLQCTRNILPDEGEPTDEETTGDISDSMDFVLLNFAEMNKLWVRMQHQ
GHSRDREKRERERQELRILVGTNLVRLSQLEGVNVERYKQIVLTGILEQV
VNCRDALAQEYLMECIIQVFPDEFHLQTLNPFLRACAELHQNVNVKNIII
ALIDRLALFAHREDGPGIPADIKLFDIFSQQVATVIQSRQDMPSEDVVSL
QVSLINLAMKCYPDRVDYVDKVLETTVEIFNKLNLEHIATSSAVSKELTR
LLKIPVDTYNNILTVLKLKHFHPLFEYFDYESRKSMSCYVLSNVLDYNTE
IVSQDQVDSIMNLVSTLIQDQPDQPVEDPDPEDFADEQSLVGRFIHLLRS
EDPDQQYLILNTARKHFGAGGNQRIRFTLPPLVFAAYQLAFRYKENSKVD
DKWEKKCQKIFSFAHQTISALIKAELAELPLRLFLQGALAAGEIGFENHE
TVAYEFMSQAFSLYEDEISDSKAQLAAITLIIGTFERMKCFSEENHEPLR
TQCALAASKLLKKPDQGRAVSTCAHLFWSGRNTDKNGEELHGGKRVMECL
KKALKIANQCMDPSLQVQLFIEILNRYIYFYEKENDAVTIQVLNQLIQKI
REDLPNLESSEETEQINKHFHNTLEHLRLRRESPESEGPIYEGLIL

The gene encoding the human retromer core protein VPS26a (Gene ID: 9559) can be used to obtain a transgene encoding a functional retromer core protein VPS26a (SEQ ID NO: 2):

MSFLGGFFGPICEIDIVLNDGETRKMAEMKTEDGKVEKHYLFYDGESVSG
KVNLAFKQPGKRLEHQGIRIEFVGQIELFNDKSNTHEFVNLVKELALPGE
LTQSRSYDFEFMQVEKPYESYIGANVRLRYFLKVTIVRRLTDLVKEYDLI
VHQLATYPDVNNSIKMEVGIEDCLHIEFEYNKSKYHLKDVIVGKIYFLLV
RIKIQHMELQLIKKEITGIGPSTTTETETIAKYEIMDGAPVKGESIPIRL
FLAGYDPTPTMRDVNKKFSVRYFLNLVLVD EEDRRYFKQQEIILWRKAP
EKLRKQRTNFH QRFESPESQASAEQPEM

The gene encoding the human retromer core protein VPS26b (Gene ID: 112936) can be used to obtain a transgene encoding a functional retromer core protein VPS26b (SEQ ID NO: 3):

MSFFGFGQSVEVEILLNDAESRKRAEHKTEDGKKEKYFLFYDGETVSGKV
SLALKNPNKRLEHQGIKIEFIGQIELYYDRGNHHEFVSLVKDLARPGEIT
QSQAFDFEFTHVEKPYESYTGQNVKLRYFLRATISRRLNDVVKEMDIVVH
TLSTYPELNSSIKMEVGIEDCLHIEFEYNKSKYHLKDVIVGKIYFLLVRI
KIKHMEIDIIKRETTGTGPNVYHENDTIAKYEIMDGAPVRGESIPIRLFL
AGYELTPTMRDINKKFSVRYYLNLVLIDEEERRYFKQQEVVLWRKGDIVR
KSMSHQAAIASQRFEGTTSLGEVRTPSQLSDNNCRQ

The wild type mice mouse mRNA sequences (i.e., coding sequences) for the retromer core proteins were obtained from National Center for Biotechnology Information (NCBI) and are set forth below.

mVPS35
(SEQ ID NO: 4)
ATGCCTACAACACAGCAGTCACCCCAGGATGAGCAGGAAAAACTTTTGGA
TGAAGCCATCCAGGCTGTGAAGGTTCAGTCATTCCAGATGAAAAGATGCC
TGGACAAAAACAAGCTGATGGATGCTCTGAAGCATGCCTC CAATATGCTT
GGAGAGCTCCGGACCTCTATGCTGTCACCAAAGAGTTACTATGAACTTTA
TATGGCTATTTCTGATGAACTGCACTACTTGGAGGTCTACCTGACTGATG
AATTTGCTAAAGGAAGAAAGGTGGCAGATCTCTATGAACTTGTACAGTAC
GCGGGAAACATTATTCCAAGGCTTTATCTCTTGATCACAGTTGGAGTTGT
ATATGTCAAGTCATTTCCTCAATCCAGGAAAGATATTTTGAAAGATTTGG
TAGAAATGTGCCGTGGTGTGCAGCATCCGCTAAGGGGTTTGTTTCTTCGA
AATTACCTTCTTCAGTGTACTAGGAACATTTTACCTGATGAAGGAGAGCC
AACAGATGAAGAAACAACTGGTGATATCAGTGATTCCATGGATTTTGTAC
TACTCAACTTTGCAGAAATGAATAAGCTCTGGGTGCGGATGCAGCATCAA
GGACATAGTCGAGATAGAGAAAAAAGAGAACGAGAGAGACAAGAACTGAG
AATTTTAGTGGGAACAAATTTGGTGCGCCTTAGTCAGTTGGAAGGTGTAA
ATGTGGAACGTTACAAACAGATTGTTCTAACAGGCATTCTGGAGCAGGTT
GTGAATTGTAGGGATGCTCTGGCTCAGGAGTATCTCATGGAGTGCATCAT
TCAGGTTTTTCCTGATGAGTTTCATCTTCAGACTTTGAATCCTTTTCTTA
GAGCTTGTGCTGAATTACACCAAAATGTAAATGTGAAAAACATAATCATT
GCTTTAATTGACAGATTAGCTTTATTTGCTCATCGTGAAGATGGACCCGG
AATTCCAGCTGAGATTAAACTTTTTGACATATTTTCACAACAGGTGGCTA
CAGTCATACAGTCCAGACAAGACATGCCATCAGAGGATGTTGTATCTTTA
CAAGTCTCTCTCATTAATCTTGCTATGAAATGTTACCCTGATCGTGTGGA
CTACGTCGATAAAGTTCTAGAAACAACAGTGGAGATATTCAATAAGCTTA
ACCTTGAACATATTGCTACCAGTAGTGCAGTTTCAAAGGAGCTTACCAGA
CTTTTGAAGATACCTGTTGATACTTACAACAATATCTTAACAGTATTAAA
GTTAAAGCATTTCCACCCACTTTTTGAGTACTTTGACTACGAGTCCAGAA
AGAGCATGAGCTGTTATGTGCTTAGTAATGTTCTGGATTATAACACAGAA
ATCGTCTCTCAGGACCAGGTAGATTCCATAATGAATTTGGTGTCCACATT
GATTCAGGATCAGCCAGACCAACCTGTAGAAGACCCTGATCCAGAAGACT
TCGCTGATGAACAGAGCCTTGTTGGCCGATTTATTCATCTGCTACGTTCT
GATGATCCTGACCAGCAGTATTTGATTTTGAATACGGCACGAAAACATTT
TGGGGCTGGTGGAAATCAGCGGATTCGCTTCACACTGCCACCTTTGGTAT
TTGCAGCTTACCAGTTGGCTTTTCGATACAAAGAGAATTCCCAAATGGAT
GACAAGTGGGAAAAGAAATGCCAGAAGATATTTTCATTTGCTCACCAGAC
TATCAGTGCTTTGATTAAAGCTGAGCTGGCTGAATTACCACTGAGACTTT
TTCTTCAAGGAGCATTAGCTGCTGGAGAAATTGGCTTTGAAAATCATGAA
ACAGTAGCATATGAATTTATGTCCCAGGCATTTTCTCTATATGAAGATGA
AATCAGTGATTCTAAAGCACAGCTGGCTGCTATCACCTTGATCATCGGTA
CTTTTGAGAGGATGAAATGCTTCAGTGAAGAGAATCATGAACCCTTGAGA
ACTCAGTGTGCACTTGCTGCATCAAAACTTCTGAAAAAACCAGATCAAGG
CCGAGCCGTGAGCACATGTGCGCATCTCTTTTGGTCTGGCCGAAACACAG
ACAAAAATGGGGAAGAGCTTCATGGAGGTAAAAGGGTCATGGAGTGCCTA
AAGAAGGCACTAAAAATAGCAAATCAGTGCATGGACCCCTCTCTACAAGT
TCAGCTCTTTATAGAGATTCTGAACAGGTACATCTATTTCTATGAAAAAG
AAAATGATGCGGTAACCATTCAAGTCTTGAACCAACTTATTCAAAAGATT
CGAGAAGATCTCCCAAATCTTGAGTCCAGTGAAGAAACAGAACAAATAAA
CAAGCATTTTCACAACACATTGGAGCACTTGCGCTCAAGACGGGAATCAC
CAGAGTCTGAGGGGCCAATCTATGAAGGTCTCATCCTTTAA
mVPS26a Isoform A
(SEQ ID NO: 5)
ATGAGTTTTCTTGGAGGCTTTTTTGGTCCCATTTGTGAGATTGATGTTGCCCTTA
ATGATGGGGAAACCAGGAAAATGGCAGAAATGAAAACTGAGGATGGCAAA
GTAGAAAAACACTATCTCTTCTATGATGGCGAGTCTGTCTCAGGAAAGGT
AAACCTAGCCTTTAAGCAGCCTGGAAAGAGGCTAGAGCATCAAGGAATTA
GAATTGAATTTGTAGGTCAAATTGAGCTTTTCAATGACAAGAGTAATACT
CATGAATTTGTAAACCTAGTGAAGGAACTAGCCTTGCCTGGAGAACTGAC
TCAGAGCAGAAGCTATGACTTTGAATTTATGCAAGTTGAAAAGCCATATG
AGTCATACATCGGTGCCAATGTCCGCCTGAGGTATTTTCTTAAGGTGACA
ATTGTGAGAAGATTGACAGACTTAGTGAAAGAGTACGATCTTATTGTTCA
TCAGCTAGCCACCTATCCTGATGTCAACAACTCTATTAAAATGGAAGTGG
GCATTGAAGACTGTCTGCACATAGAGTTTGAATATAATAAGTCCAAGTAT
CATTTAAAGGATGTAATTGTTGGAAAAATTTACTTCTTATTAGTAAGAAT
AAAAATACAACACATGGAATTACAGCTGATCAAGAAAGAGATCACAGGAA
TTGGACCCAGCACCACAACAGAGACAGAAACAATCGCTAAGTATGAAATA
ATGGATGGGGCGCCAGTAAAAGGAGAATCTATTCCGATAAGATTGTTCTT
AGCAGGGTATGACCCAACCCCCACGATGAGAGATGTGAACAAGAAGTTTT
CAGTAAGGTACTTTCTAAACCTCGTGCTTGTTGATGAGGAGGACCGAAGG
TACTTCAAGCAGCAGGAGATCATCCTGTGGAGAAAAGCACCCGAGAAACT
GAGAAAACAGAGGACGAACTTTCACCAGCGGTTTGAATCTCCAGACTCGC
AGGCCTCTGC GGAGCAGCCT GAGATGTAA
mVPS26b
(SEQ ID NO: 6)
ATGAGCTTCTTCGGCTTCGGGCAGAGCGTGGAGGTGGAAATCTTGCTGAATGATGCG
GAGAGTAGGAAGCGAGCGGAGCACAAGACTGAGGACGGGAAGAAGGAGAAATATT
TCCTCTTCTACGACGGGGAAACCGTCTCGGGGAAAGTGAGCCTATCACTGAAGAAC
CCCAACAAGCGACTAGAGCACCAGGGCATCAAGATCGAGTTCATTGGGCAGATCGA
ACTCTACTATGACCGTGGGAACCACCATGAGTTTGTGTCTTTGGTGAAGGACCTGGC
TCGGCCAGGAGAGATCACCCAATCGCAGGCCTTCGACTTTGAGTTCACCCATGTGGA
AAAGCCGTATGAATCCTACACAGGACAGAATGTGAAGCTCCGCTATTTCCTTCGAGC
CACCATCAGCCGCCGCCTCAATGATGTTGTTAAAGAGATGGACATTGTAGTTCACAC
ACTTAGCACATACCCCGAGCTGAACTCATCCATCAAGATGGAAGTTGGCATTGAGG
ATTGCCTGCACATTGAATTTGAGTACAACAAATCCAAATACCACTTGAAAGATGTCA
TTGTAGGGAAGATATACTTCCTGCTGGTGAGAATCAAGATCAAGCACATGGAGATA
GACATCATCAAACGAGAGACAACAGGCACGGGTCCCAACGTGTACCACGAGAACG
ACACAATAGCGAAGTATGAGATCATGGACGGGGCACCAGTCCGAGGTGAGTCCATC
CCCATCAGGCTCTTCCTGGCAGGGTATGAGCTCACACCCACCATGCGTGACATAAAT
AAGAAGTTCTCTGTGCGCTATTACCTCAACTTGGTGCTGATAGATGAGGAGGAACGG
CGCTACTTCAAGCAGCAGGAAGTGGTGTTGTGGCGGAAGGGTGACATCGTACGGAA
GAGCATGTCCCACCAGGCAGCCATTGCCTCACAGCGCTTCGAGGGCACAACCTCCCT
GGGTGAGGTGCGGACCCCTGGCCAACTGTCTGACAACAACAGCAGGCAGTAG

### Codon Optimization of Transgene Coding Sequences

Codon optimization of the transgene coding sequences can increase the efficiency of the gene therapy. Thus, in some embodiments, a nucleic acid that is at least 70% identical to the coding sequence of the transgene encoding the therapeutic protein (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence) is used.

Codon optimization tools are known in the art.

Exemplary codon optimized nucleic acids are as follows.

mVPS35 Codon optimized
(SEQ ID NO: 7)
ATGCCCACAACACAGCAGAGCCCACAGGATGAACAGGAAAAGCTGCTGGATGAGG
CCATCCAGGCTGTGAAAGTGCAGTCCTTCCAGATGAAAAGATGTCTGGATAAGAAT
AAGCTGATGGACGCCCTGAAGCATGCCAGCAACATGCTGGGCGAGCTGAGGACCAG
CATGCTGTCTCCCAAGTCCTACTACGAGCTGTACATGGCCATTTCTGACGAGCTGCA
CTACCTGGAGGTGTACCTCACAGATGAGTTCGCCAAGGGGAGAAAGGTGGCCGATC
TGTATGAGCTGGTGCAGTATGCTGGCAACATCATCCCCAGGCTGTACCTGCTGATCA
CCGTGGGCGTGGTGTATGTGAAGAGCTTCCCCCAGTCTAGAAAGGATATCCTGAAG
GACCTGGTGGAAATGTGCAGGGGAGTGCAGCATCCTCTGAGAGGCCTGTTTCTGAG
AAACTACCTGCTGCAGTGTACCAGGAATATCCTGCCCGACGAAGGCGAGCCTACTG
ATGAAGAAACAACCGGCGACATCAGCGACAGCATGGATTTTGTGCTGCTGAACTTC
GCCGAGATGAACAAACTGTGGGTGAGAATGCAGCACCAGGGACACAGCAGGGACA
GAGAGAAGAGAGAGAGGGAGAGACAGGAGCTGAGAATCCTGGTGGGCACCAACCT
GGTGAGGCTGTCCCAGCTGGAGGGCGTGAATGTGGAGAGATATAAGCAGATTGTGC
TGACAGGAATCCTGGAGCAGGTGGTGAACTGCAGAGATGCCCTGGCCCAGGAGTAT
CTGATGGAATGCATCATTCAGGTGTTCCCAGATGAGTTCCACCTGCAGACACTGAAC
CCTTTCCTGAGGGCCTGCGCCGAGCTGCATCAGAACGTGAACGTCAAGAACATCATC
ATTGCTCTGATTGACAGGCTGGCCCTGTTTGCCCATAGAGAGGATGGACCTGGCATC
CCAGCCGAGATCAAGCTGTTTGACATTTTCAGCCAGCAGGTGGCCACCGTGATCCAG
TCTAGGCAGGACATGCCTTCCGAAGATGTGGTGAGCCTGCAGGTGTCCCTGATCAAT
CTGGCCATGAAGTGTTACCCTGATAGAGTGGATTACGTGGACAAGGTGCTGGAGAC
CACAGTGGAGATCTTCAACAAGCTGAACCTGGAGCACATCGCCACTTCTTCTGCTGT
GTCTAAAGAACTGACAAGACTGCTGAAGATCCCTGTGGACACCTACAACAACATTCT
GACCGTGCTGAAGCTGAAACACTTCCATCCACTGTTCGAGTACTTTGATTATGAGAG
CAGGAAGTCCATGAGCTGCTACGTGCTGAGCAATGTGCTGGACTACAATACAGAAA
TCGTGTCCCAGGATCAGGTGGACAGCATCATGAATCTGGTGAGCACACTGATCCAG
GACCAGCCTGATCAGCCAGTGGAGGACCCCGACCCTGAGGACTTCGCTGACGAGCA
GAGCCTGGTGGGCAGGTTCATCCACCTGCTGAGGTCTGATGATCCAGACCAGCAGTA
CCTGATCCTGAACACCGCCAGGAAGCACTTTGGAGCCGGAGGCAACCAGAGGATCA
GATTCACCCTGCCTCCCCTGGTGTTTGCCGCCTACCAGCTGGCTTTCAGATACAAGG
AAAATTCCCAGATGGATGACAAGTGGGAGAAAAAATGCCAGAAGATCTTTTCTTTT
GCTCACCAGACCATCTCTGCCCTGATCAAAGCTGAGCTGGCTGAACTGCCACTGAGA
CTGTTCCTCCAGGGGGCCCTGGCTGCCGGGGAGATCGGCTTTGAGAACCATGAGACT
GTGGCCTATGAATTCATGTCTCAGGCCTTCTCTCTGTACGAAGATGAGATCAGCGAC
AGCAAGGCTCAGCTGGCTGCTATCACACTGATCATTGGAACATTTGAAAGGATGAA
GTGCTTCTCCGAGGAAAACCACGAGCCACTGAGAACACAGTGTGCACTGGCCGCCT
CCAAGCTGCTGAAGAAGCCTGACCAGGGCAGAGCCGTGTCTACCTGTGCCCACCTG
TTCTGGAGCGGAAGAAACACAGACAAAAATGGAGAGGAGCTGCATGGCGGCAAGA
GGGTGATGGAGTGCCTGAAGAAAGCCCTGAAGATTGCCAACCAGTGCATGGACCCA
AGCCTGCAGGTGCAGCTGTTCATCGAGATCCTGAACAGGTACATCTACTTCTACGAG
AAGGAGAATGACGCCGTGACCATTCAGGTGCTGAATCAGCTGATCCAGAAAATCAG
GGAAGACCTGCCCAACCTGGAGAGCTCTGAAGAGACAGAGCAGATCAACAAGCATT
TTCACAACACTCTGGAACATCTGAGGTCCAGGAGGGAGTCCCCAGAGTCTGAGGGA
CCCATCTATGAGGGGCTGATCCTGTGA
mVPS26a, Isoform A, Codon optimized
(SEQ ID NO: 8)
ATGAGCTTCCTGGGCGGCTTCTTCGGCCCCATCTGTGAGATCGACGTGGCCCTGAAC
GACGGCGAGACCAGAAAGATGGCCGAGATGAAGACAGAGGATGGCAAGGTGGAGA
AGCACTACCTGTTCTACGACGGAGAGTCTGTGTCCGGCAAGGTGAACCTGGCCTTCA
AGCAGCCTGGGAAGAGGCTGGAGCACCAGGGCATCAGAATCGAGTTCGTGGGCCAG
ATCGAGCTGTTCAACGACAAGAGCAACACCCACGAGTTTGTGAACCTGGTGAAGGA
GCTGGCTCTGCCTGGCGAGCTGACCCAGAGCAGAAGCTACGACTTCGAGTTCATGC
AGGTGGAGAAGCCTTACGAGAGCTACATCGGCGCCAACGTGAGACTGAGATACTTC
CTGAAGGTGACCATCGTGAGGAGACTGACCGACCTGGTGAAGGAGTATGACCTGAT
CGTGCACCAGCTGGCCACCTACCCTGACGTGAACAACAGCATCAAGATGGAGGTGG
GCATCGAGGACTGCCTGCACATCGAGTTCGAGTACAACAAGTCCAAGTACCACCTG
AAGGACGTGATCGTGGGCAAGATCTACTTCCTGCTGGTGAGGATCAAGATCCAGCA
CATGGAGCTGCAGCTGATCAAGAAGGAGATCACCGGCATCGGCCCTTCCACAACCA
CCGAGACAGAGACAATCGCCAAGTACGAGATCATGGACGGCGCCCCTGTGAAGGGC
GAGAGCATCCCTATCAGGCTGTTCCTGGCCGGCTACGACCCTACCCCTACCATGAGA
GACGTGAACAAGAAGTTCAGCGTGAGGTACTTCCTGAACCTGGTGCTGGTGGACGA
GGAGGACAGAAGATACTTCAAGCAGCAGGAGATCATCCTGTGGAGGAAGGCCCCTG
AGAAGCTGAGGAAGCAGAGGACCAACTTCCACCAGAGATTCGAGTCCCCTGACAGC
CAGGCCAGCGCCGAGCAGCCAGAGATGTGA
mVPS26b Codon optimized
(SEQ ID NO: 9)
ATGAGTTTTTTTGGGTTTGGACAGTCAGTGGAGGTGGAGATCCTGCTGAACGACGCC
GAGAGCAGGAAGAGGGCCGAGCACAAGACAGAGGATGGCAAGAAGGAGAAGTACT
TCCTGTTCTACGACGGAGAGACCGTGAGCGGCAAGGTGTCCCTGAGCCTGAAGAAC
CCAAACAAGAGACTGGAGCACCAGGGCATCAAGATCGAGTTCATCGGCCAGATCGA
GCTGTACTACGACAGGGGCAACCACCACGAGTTCGTGAGCCTGGTGAAGGACCTGG
CCAGACCTGGCGAGATCACCCAGAGCCAGGCCTTCGACTTCGAGTTCACCCACGTG
GAGAAGCCTTACGAGAGCTACACAGGCCAGAACGTGAAGCTGAGGTACTTCCTGAG
AGCCACCATCAGCAGAAGACTGAACGACGTGGTGAAGGAGATGGACATCGTGGTGC
ACACCCTGAGCACCTACCCTGAGCTGAACTCTTCCATCAAGATGGAGGTGGGCATCG
AGGACTGCCTGCACATCGAGTTCGAGTACAACAAGAGCAAGTACCACCTGAAGGAC
GTGATCGTGGGCAAGATCTACTTCCTGCTGGTGAGAATCAAGATCAAGCACATGGA
GATCGACATCATCAAGAGAGAGACCACAGGCACAGGCCCTAACGTGTACCACGAGA
ACGACACCATCGCCAAGTACGAGATCATGGACGGCGCCCCTGTGAGAGGCGAGAGC
ATCCCTATCAGGCTGTTCCTGGCCGGCTACGAGCTGACCCCTACCATGAGGGACATC
AACAAGAAGTTCAGCGTGAGATACTACCTGAACCTGGTGCTGATCGATGAGGAGGA
GAGAAGATACTTCAAGCAGCAGGAGGTGGTGCTGTGGAGAAAGGGCGACATCGTGA
GAAAGAGCATGAGCCACCAGGCCGCCATCGCCAGCCAGAGATTCGAGGGCACCACC
AGCCTGGGCGAGGTGAGGACCCCTGGCCAGCTGAGCGACAACAACAGCAGACAGT
GA
Human VPS 3 5 coding sequence (with certain codons modified to remove
restriction sites)
(SEQ ID NO: 10)
ATGCCTACAACACAGCAGTCCCCTCAGGATGAGCAGGAAAAGCTCTTGGATGAAGC
CATACAGGCTGTGAAGGTCCAGTCATTCCAAATGAAGAGATGCCTGGACAAAAACA
AGCTCATGGATGCTCTAAAACATGCTTCTAATATGCTTGGTGAACTCCGGACTTCTA
TGTTATCACCAAAGAGTTACTATGAACTTTATATGGCCATTTCTGATGAACTGCACT
ACTTGGAGGTCTACCTGACAGATGAGTTTGCTAAAGGAAGGAAAGTGGCAGATCTC
TACGAACTTGTACAGTATGCTGGAAACATTATCCCAAGGCTTTACCTTCTGATCACA
GTTGGAGTTGTATATGTCAAGTCATTTCCTCAGTCCAGGAAGGATATTCTGAAAGAT
TTGGTAGAAATGTGCCGTGGTGTGCAACATCCCTTGAGGGGTCTGTTTCTTCGAAAT
TACCTTCTTCAGTGTACCAGAAATATCTTACCTGATGAAGGAGAGCCAACAGATGAA
GAAACAACTGGTGACATCAGTGATTCCATGGATTTCGTACTGCTCAACTTTGCAGAA
ATGAACAAGCTCTGGGTGCGAATGCAGCATCAGGGACATAGCCGAGATAGAGAAAA
AAGAGAACGAGAAAGACAAGAACTGAGGATTCTAGTGGGAACAAATTTGGTGCGCC
TCAGTCAGTTGGAAGGTGTAAATGTGGAACGTTACAAACAGATTGTTCTGACTGGCA
TATTGGAGCAAGTTGTAAACTGTAGGGATGCTTTGGCTCAAGAATATCTCATGGAGT
GTATTATTCAGGTATTCCCTGATGAATTTCACCTCCAGACTTTGAATCCTTTCCTTCG
GGCCTGTGCTGAGTTACACCAGAATGTAAATGTGAAGAACATAATCATTGCTTTAAT
TGATAGATTAGCTTTATTTGCTCACCGTGAAGATGGACCTGGAATCCCAGCGGATAT
TAAACTATTCGATATATTCTCACAGCAGGTGGCTACAGTGATACAGTCAAGACAAGA
CATGCCTTCAGAGGATGTTGTATCTTTACAAGTATCTCTTATTAATCTTGCCATGAAA
TGTTACCCTGATCGTGTGGACTATGTTGATAAAGTACTAGAAACAACAGTGGAGATA
TTCAATAAGCTCAACCTTGAACATATTGCTACCAGTAGTGCAGTTTCAAAGGAACTC
ACCAGACTATTGAAAATACCAGTTGACACTTACAACAATATATTAACAGTCTTGAAA
TTAAAACATTTCCACCCACTCTTTGAGTACTTTGACTACGAGTCCAGAAAGAGCATG
AGTTGTTATGTGCTTAGTAATGTTCTGGATTATAACACAGAAATTGTATCTCAAGAC
CAGGTGGATTCCATAATGAATTTGGTATCCACGTTGATTCAAGATCAGCCAGATCAA
CCTGTAGAAGACCCTGATCCAGAAGATTTCGCTGATGAGCAGAGCCTTGTGGGCCGC
TTCATTCATCTGCTGCGCTCTGAGGACCCTGACCAGCAGTACTTGATATTGAACACA
GCACGAAAACATTTCGGAGCTGGTGGAAATCAGCGGATTCGCTTCACACTGCCACCT
TTGGTATTTGCAGCTTACCAGCTGGCATTTCGATATAAAGAGAACTCTAAAGTGGAT
GACAAATGGGAAAAGAAATGCCAGAAGATATTCTCATTTGCCCACCAGACTATCAG
TGCTTTGATCAAAGCAGAGCTGGCAGAATTGCCGTTAAGACTATTCCTTCAAGGAGC
ACTAGCTGCTGGGGAAATTGGATTTGAAAATCATGAAACAGTCGCATATGAATTCAT
GTCCCAGGCATTCTCTCTGTATGAAGATGAAATCAGCGATTCCAAAGCACAGCTTGC
TGCCATCACCTTGATCATTGGCACATTTGAAAGGATGAAGTGCTTCAGTGAAGAGAA
TCATGAACCTCTGAGGACTCAGTGTGCCCTTGCTGCATCCAAACTTCTAAAGAAACC
TGATCAGGGCCGAGCTGTGAGCACCTGTGCACATCTCTTCTGGTCTGGCAGAAACAC
GGACAAAAATGGGGAGGAGCTTCACGGAGGCAAGAGGGTAATGGAGTGCCTAAAA
AAAGCTCTAAAAATAGCAAATCAGTGCATGGACCCCTCTCTACAAGTGCAGCTATTC
ATAGAAATTCTGAACAGATATATCTACTTCTATGAAAAGGAAAATGATGCGGTAAC
AATTCAGGTATTAAACCAGCTTATCCAAAAGATTCGAGAAGACCTCCCGAATCTTGA
ATCCAGTGAAGAAACAGAGCAGATTAACAAACACTTTCATAACACACTGGAGCATT
TGCGCTTGCGGCGGGAATCACCAGAATCCGAGGGGCCAATTTATGAAGGACTCATC
CTTTAA

### Routes of Administration and Dosing

The current disclosure provides rAAV vectors for use in methods of treating, preventing, and/or curing a neurodegenerative disease or disorder and/or alleviating in a subject at least one of the symptoms associated with a neurodegenerative disease and/or disorder. In some embodiments, methods involve administration of a rAAV vector that encodes one or more therapeutic polypeptides or proteins, in a pharmaceutically-acceptable carrier to the subject in an amount and for a period of time sufficient to treat, prevent and/or cure the neurodegenerative disease or disorder in the subject having or suspected of having such a neurodegenerative disease or disorder.

The rAAV vectors may be delivered to a subject in compositions according to any appropriate methods known in the art. The rAAV vector, preferably suspended in a physiologically compatible carrier (e.g., in a composition), may be administered to a subject. In certain embodiments, compositions may comprise a rAAV vector alone, or in combination with one or more other vectors (e.g., a second rAAV vector having one or more different transgenes). In one embodiment, a composition can comprise an rAAV9 vector comprising a nucleic acid sequence comprising a transgene encoding a functional protein including but not limited to retromer core protein VPS35 and/or retromer core protein VPS26a and/or retromer core protein VPS26b. In one embodiment, a composition can comprise an rAAV2/9 vector comprising a nucleic acid sequence comprising a transgene encoding a functional protein including but not limited to retromer core protein VPS35 and/or retromer core protein VPS26a and/or retromer core protein VPS26b. In one embodiment, a composition can comprise an rAAV10 or rAAV2/10 vector comprising a nucleic acid sequence comprising a transgene encoding a functional protein including but not limited to retromer core protein VPS35 and/or retromer core protein VPS26a and/or retromer core protein VPS26b.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions disclosed herein may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

In some embodiments, rAAV compositions are formulated to reduce aggregation of AAV particles in the composition, particularly where high rAAV concentrations are present (e.g., about 10¹³ GC/ml or more). Methods for reducing aggregation of rAAVs are well known in the art and, include, for example, addition of surfactants, pH adjustment, and salt concentration adjustment (see, e.g., Wright, et al., Molecular Therapy 12:171-178 (2005).

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations, transdermal matrices (U.S. Pat. Nos. 5,770,219 and 5,783,208) and feedback-controlled delivery (U.S. Pat. No. 5,697,899).

rAAVS are administered by a route of administration and in sufficient amounts to transfect the cells of a desired tissue and to provide sufficient levels of gene transfer and expression without undue adverse effects. Conventional and pharmaceutically acceptable routes of administration include, but are not limited to, direct delivery to the selected tissue (e.g., intracerebral administration, intrathecal administration), intravenous, oral, inhalation (including intranasal and intratracheal delivery), intraocular, intravenous, intramuscular, subcutaneous, intradermal, and other parental routes of administration. Routes of administration may be combined, if desired. The administration regimen depends on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects. Accordingly, the amount of biologic delivered depends in part on the particular therapeutic composition and the severity of the condition being treated.

The present disclosure provides stable pharmaceutical compositions comprising rAAV virions. The compositions remain stable and active even when subjected to freeze/thaw cycling and when stored in containers made of various materials, including glass.

Appropriate doses will depend on the subject being treated (e.g., human or nonhuman primate or other mammal), age and general condition of the subject to be treated, the severity of the condition being treated, the mode of administration of the rAAV virions, among other factors. An appropriate effective amount can be readily determined by one of skill in the art.

The dose of rAAV virions required to achieve a desired effect or “therapeutic effect,” e.g., the units of dose in vector genomes/per kilogram of body weight (vg/kg), will vary based on several factors including, but not limited to: the route of rAAV administration; the level of gene or RNA expression required to achieve a therapeutic effect; the specific disease or disorder being treated; and the stability of the gene or RNA product. One of skill in the art can readily determine a rAAV virion dose range to treat a subject having a particular disease or disorder based on the aforementioned factors, as well as other factors that are well known in the art. An effective amount of the rAAV is generally in the range of from about 10 μl to about 100 ml of solution containing from about 10⁹ to 10¹⁶ genome copies per subject. Other volumes of solution may be used. The volume used will typically depend, among other things, on the size of the subject, the dose of the rAAV, and the route of administration. For example, for intrathecal or intracerebral administration a volume in range of 1 μl to 10 μl or 10 μl to 100 μl may be used. For intravenous administration a volume in range of 10 μl to 100 μl, 100 μl to 1 ml, 1 ml to 10 ml, or more may be used. In some cases, a dosage between about 10¹⁰ to 10¹² rAAV genome copies per subject is appropriate. In certain embodiments, 10¹² rAAV genome copies per subject is effective to target desired tissues. In some embodiments the rAAV is administered at a dose of 10¹⁰, 10¹¹, 10¹², 10¹³, 10¹⁴, or 10¹⁵ genome copies per subject. In some embodiments the rAAV is administered at a dose of 10¹⁰, 10¹¹, 10¹², 10¹³, or 10¹⁴ genome copies per kg.

Thus, a “therapeutically effective amount” will fall in a relatively broad range that can be determined through clinical trials. For example, for in vivo injection, i.e., injection directly to the subject, a therapeutically effective dose may be on the order of from about 10⁵ to 10¹⁶ of the rAAV virions, more preferably 10⁸ to 10¹⁴ rAAV virions. For in vitro transduction, an effective amount of rAAV virions to be delivered to cells may be on the order of 10⁵ to 10¹³, preferably 10⁸ to 10¹³ of the rAAV virions. If the composition comprises transduced cells to be delivered back to the subject, the amount of transduced cells in the pharmaceutical compositions may be from about 10⁴ to 10¹⁰ cells, more preferably 10⁵ to 10⁸ cells. The dose, of course, depends on the efficiency of transduction, promoter strength, the stability of the message and the protein encoded thereby. Effective dosages can be readily established by one of ordinary skill in the art through routine trials establishing dose response curves.

Dosage treatment may be a single dose schedule or a multiple dose schedule to ultimately deliver the amount specified above. Moreover, the subject may be administered as many doses as appropriate. Thus, the subject may be given, e.g., 10⁵ to 10¹⁶ rAAV virions in a single dose, or two, three, four, five, six or more doses that collectively result in delivery of, e.g., 10⁵ to 10¹⁶ rAAV virions. One of skill in the art can readily determine an appropriate number of doses to administer.

Pharmaceutical compositions will thus comprise sufficient genetic material to produce a therapeutically effective amount of the protein of interest, i.e., an amount sufficient to reduce or ameliorate symptoms of the disease state in question or an amount sufficient to confer the desired benefit. Thus, rAAV virions will be present in the subject compositions in an amount sufficient to provide a therapeutic effect when given in one or more doses. The rAAV virions can be provided as lyophilized preparations and diluted in the virion-stabilizing compositions for immediate or future use. Alternatively, the rAAV virions may be provided immediately after production and stored for future use.

The pharmaceutical compositions will also contain a pharmaceutically acceptable excipient or carriers. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences and U.S. Pharmacopeia: National Formulary, Mack Publishing Company, Easton, Pa. (1984).

Formulations of therapeutic and diagnostic agents may be prepared by mixing with acceptable carriers, excipients, or stabilizers in the form of, e.g., lyophilized powders, slurries, aqueous solutions or suspensions.

Toxicity and therapeutic efficacy of the therapeutic compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, therapeutic compositions exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

Determination of the appropriate dose is made by the clinician, e.g., using parameters or factors known or suspected in the art to affect treatment. The dose may begin with an amount somewhat less than the optimum dose and it may be increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent.

A preferred route of administration of the AAVs is intravenously. Other routes of administration of the rAAV vectors described herein include intracranial, intraparenchymal, intraspinal.

A preferred dose ranges from about 1×10¹⁰) to about 8×10¹¹, from about 2×10¹⁰ to about 6×10¹¹, from about 4×10¹⁰ to about 4×10¹¹ genome or viral copy (vc) total administration. A preferred dose is about 4×10¹¹ genome or viral copy (vc) total administration of rAAV.

If more than one rAAV is used a preferred total dose of vector ranges from about 1×10¹⁰ to about 6×10¹⁰, from about 2×10¹⁰ to about 5×10¹¹, from about 1×10¹⁰ to about 4×10¹¹ genome or viral copy (vc) total administration. A preferred dose of total vector is about 3×10¹¹. The AAV can be administered in equal amounts, e.g., ratio of 50/50, or in or in ratios of about 5/95, 10/90, 15/85, 20/80, 25/75, 30/70, 35/65, 40/60, 45/55, 55/45, 60/40, 65/35, 70/30, 75/25, 80/20, 85/15, 90/10, and 95/5.

Doses can be adjusted to optimize the effects in the subject. Additionally, a subject can be monitored for improvement of their condition prior to increasing the dosage. A subject's response to the therapeutic administration of the rAAV can be monitored by observing a subject's muscle strength and control, and mobility as well as changes in height and weight. If one or more of these parameters increase after the administration, the treatment can be continued. If one or more of these parameters stays the same or decreases, the dosage can be increased.

### Kits

The present disclosure also provides kits comprising the components of the combinations disclosed herein in kit form. A kit of the present disclosure includes one or more components including, but not limited to, viral vectors (e.g., AAV vectors)) described herein. Kits may further include a pharmaceutically acceptable carrier, as discussed herein. The viral vector can be formulated as a pure composition or in combination with a pharmaceutically acceptable carrier, in a pharmaceutical composition.

In some embodiments, a kit includes an AAV vector containing a transgene described herein in one container (e.g., in a sterile glass or plastic vial).

In some embodiments, a kit includes an AAV vector containing a transgene described herein in one container (e.g., in a sterile glass or plastic vial) and a second AAV vector encoding a transgene described herein in another container (e.g., in a sterile glass or plastic vial) and a third AAV vector encoding a transgene described herein in another container (e.g., in a sterile glass or plastic vial).

In some embodiments, a kit includes an AAV vector encoding retromer core protein VPS35 and/or retromer core protein VPS26a and/or retromer core protein VPS26b, or a pharmaceutical composition thereof in one or more containers (e.g., in a sterile glass or plastic vial).

If the kit includes one or more pharmaceutical compositions for parenteral administration to a subject, the kit can include a device for performing such administration. For example, the kit can include one or more hypodermic needles or other injection devices as discussed above.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

### EXAMPLES

The present invention may be better understood by reference to the following non-limiting examples, which are presented in order to more fully illustrate the preferred embodiments of the invention. They should in no way be construed to limit the broad scope of the invention.

### Example 1- Materials and Methods

Plasmid Production

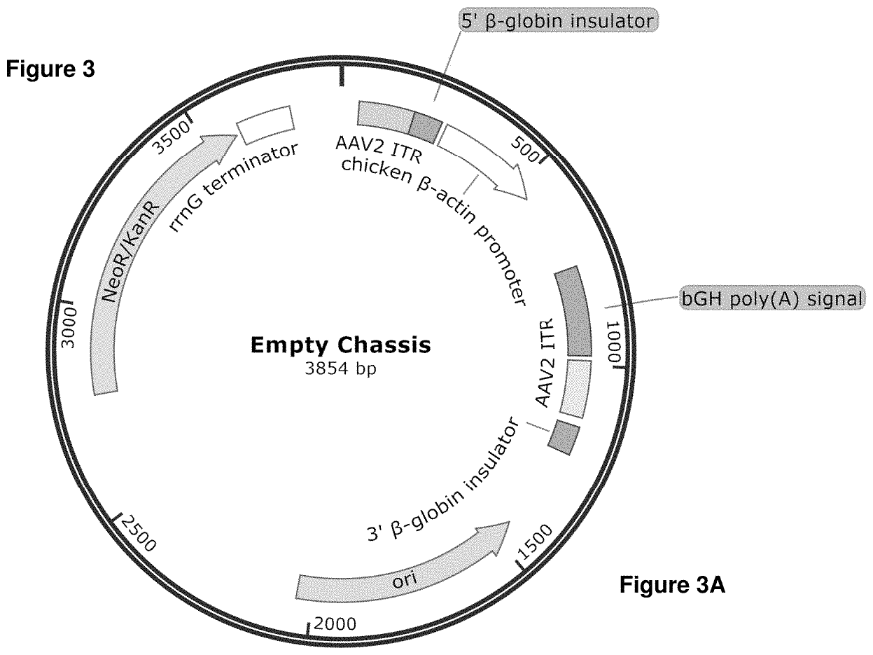

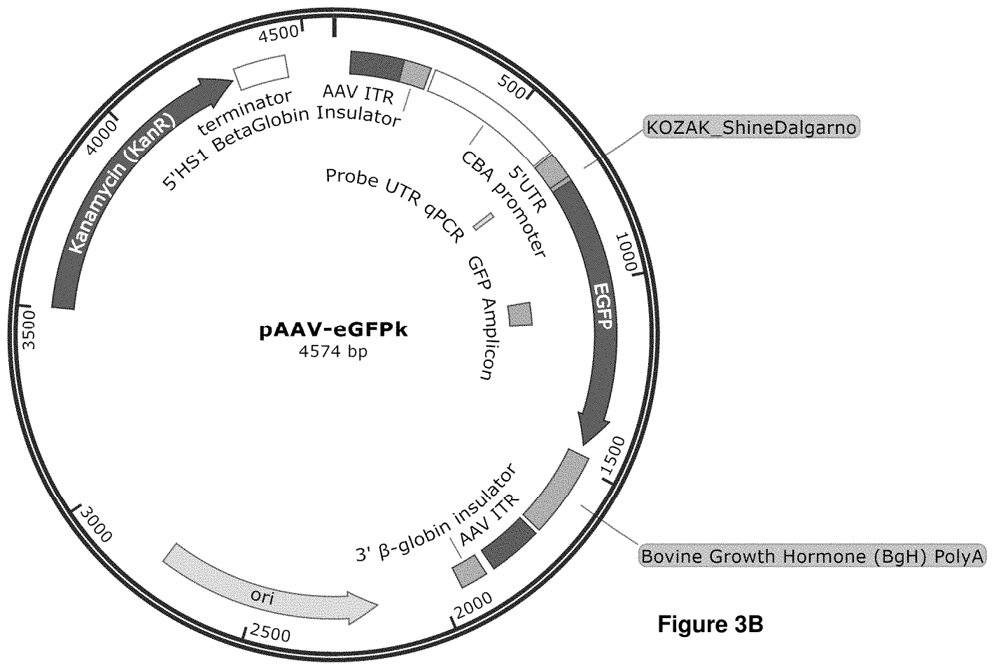

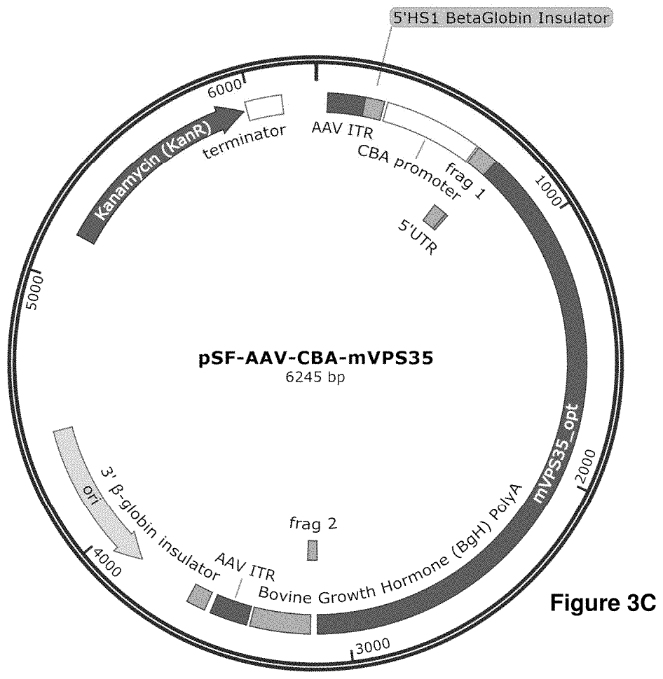

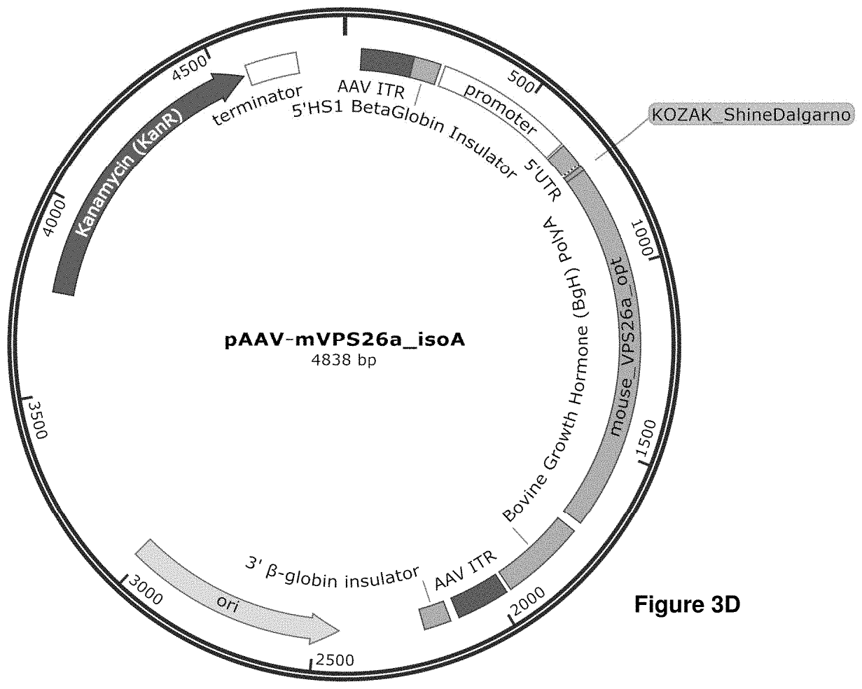

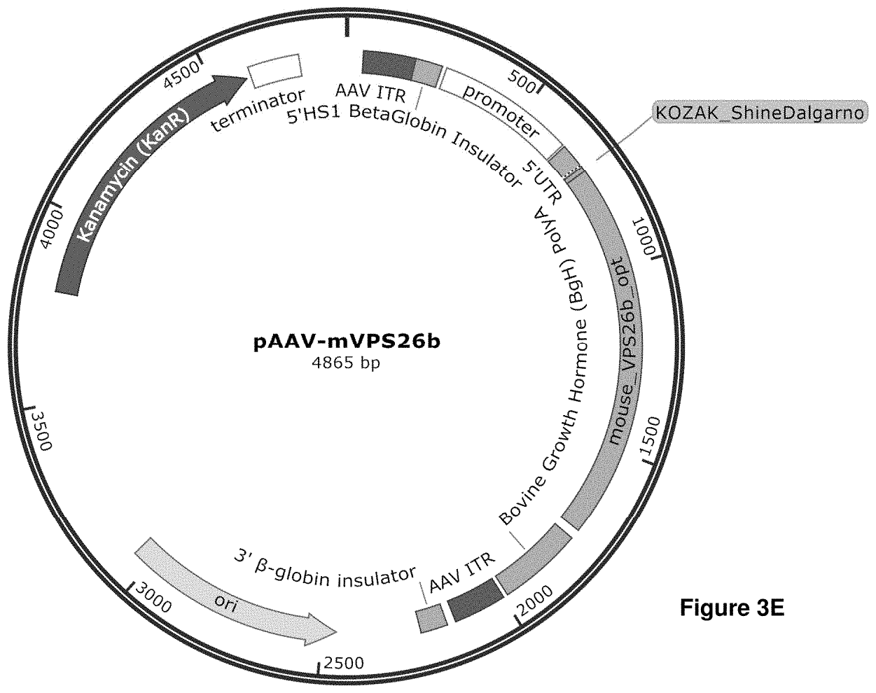

mRNA sequences for VPS35, VPS26a and VPS26b were acquired from the National Center for Biotechnology Information (NCBI). The sequences were codon optimized and then de novo synthesized. The synthesized construct was sub-cloned into AAV transfer plasmid with AAV2 inverted terminal repeats (ITRs) and a ubiquitous Chicken beta Actin wild type promoter. The transgene was followed by the bovine growth hormone polyadenylation signal. Empty chassis vector control was generated by deleting the VPS35 sequence. The GFP control was designed to mimic the rationale design of the target VPS-constructs. It contained an enhanced green flourescent protein, the same Bovine Growth Hormone polyA (BgH) and Chicken beta Actin wild type promoter, as the VPS constructs. The resulting plasmids are shown in FIG. 3.

### AAV9 Production

Each of the constructs described above were individually packaged into a recombinant adeno-associated virus vector 9 (AAV9), using capsid and helper plasmid DNA from MeiraGTx. Briefly the transfer plasmid, rep-cap plasmid and helper plasmids were co-transfected into HEK 293 cells. The harvested suspension containing virus and cellular debris was clarified using millipore SHC XL150 filter (140 cm²). The clarified suspension was then purified with AVB Sepharose, 20 mL column, elution in 3 column volumes. Concentration and diafiltration was performed with 100 kD mPES hollow fibre (Spectrum MicroKros cat #CO2-E100-05-S). Further concentration was done with Amicon Ultra-4 Centrifugal Filter 30 kD (cat #UFC8030).

### N2A Culture

Mouse neuroblastoma (N2a) cells were cultured in 50% DMEM (high glucose) and 50% Opti-MEM+10% FBS and Glutamine (2 mM) with penicillin and streptomycin to prevent microbial contamination.

### Transfection

Lipofectamine transfection protocols were used with some modifications. Briefly lipofectamine LTX was used to co-transfect Vps35 and Vps26 (Vps26a or Vps26b) plasmids into neuron like cells, Neuro2a (N2a) in a 6 well format. 100k cells were plated in each well already containing medium with DNA-lipofectamine complexes. Empty chassis and GFP as were used as control plasmids. Amount of DNA copies introduced per well was 2.81E+11. Cells were harvested 48 hours after transfection using RIPA buffer as described previously (Qureshi et al. 2019).

### Neuronal Culture and Transduction

Primary mouse cortical and hippocampal neuronal cultures implemented as described previously (Bhalla et al. 2012). Neurons (450k cells per well) were transduced with retromer AAV9 (2.27E+10 vector genomes per condition per well), 7 days after plating in a 12 well plate. Empty chassis AAV9 and GFP AAV9 were used as controls. The cultures were maintained for 3 weeks after transduction (4 weeks total). At day 28 the neurons were lysed using RIPA buffer with protease and phosphatase inhibitors.

### Western Blots

Cells from N2A and neuronal cultures were lysed in RIPA and protein were isolated as described previously (Qureshi et al. 2019; Kirby et al. 2015)). Lysates from the samples were run on NuPAGE® Bis-Tris 4-12% gels, transferred onto nitrocellulose membranes using iblot and were probed with antibodies.

Primary antibodies targeting the following proteins were used: VPS35 (ab57632, Abcam, 1:1k); VPS26a (ab211530, Abcam, 1:500); VPS26b (NBP1-92575, Novus, 1:500 or 15915-1-AP, Proteintech, 1:500); VPS29 (sab2501105, Sigma-Aldrich, 1:500); Sorll (611861, BD-biosciences, 1:2k and 793 22, Cell Signaling, 1:500); and β-actin (ab6276, Abcam, 1:5k). IRDye® 800 or 680 antibodies (LI-COR) were used as secondary with dilutions of 1:10k for 800CW, 1:15k for 680RD, and 1:25k for 680LT antibodies. Western blots were scanned using the Odyssey imaging system as described previously (Eaton et al. 2013).

For Sorll (BD-611861) Peroxidase AffiniPure Donkey Anti-Mouse IgG (H+L) was used as secondary antibody (Jackson Immuno Research labs, 1:2k), and blots scanned at Fujifilm LAS-3000 Imager.

### Statistics

Statistical analysis was performed using Microsoft Excel and SPSS. Independent two-sample student's t-test, assuming equal variance, with two-tailed distribution was used for all experiments unless stated otherwise. All data are presented as means, the error bars indicate standard error of the mean. All bar graphs were created in GraphPad Prism 8. Scatter plots were created in SPSS.

### Example 2—VPS35 Expression Alone is Insufficient to Elevate Retromer's Trimer and Function

To determine the effect exogenous VPS35 overexpression has on retromer core proteins and on retromer function in a non-deficient state, cultured wild-type mouse neurons were transduced with AAV9-VPS35-HA and used either AAV9-GFP or AAV9-empty vector (EV) as control conditions, and harvested 3 weeks later.

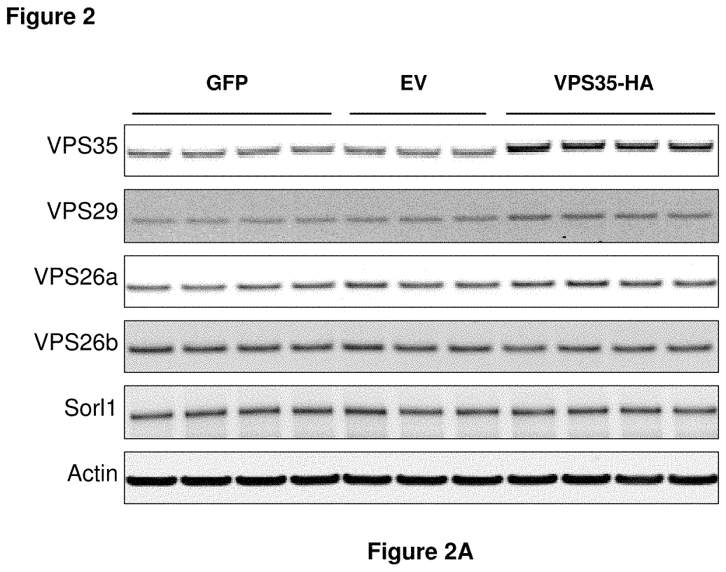

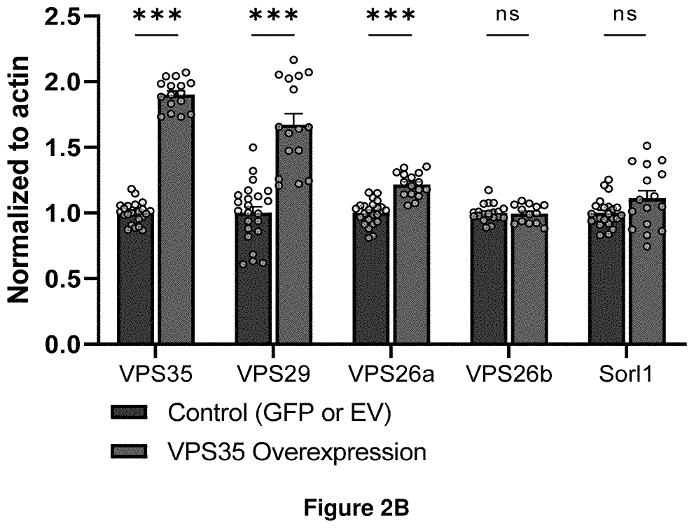

The levels of all retromer core proteins were determined by immunoblotting (FIG. 2A). Compared to controls, a 90% VPS35-HA overexpression leads to a robust 67% increase in endogenous VPS29 (p=9E-09), a small 22% increase in VPS26a (p=2E-08), and no increase in VPS26b (p=0.62) (FIG. 2B).

Sorll levels were also determined by immunoblotting. Compared to controls, overexpression of VPS35 alone had a slight (11%) and statistically unreliable (p=0.06) increase in Sorll (FIG. 2B).

By showing that VPS35 overexpression leads to a robust overexpression of VPS29, but either no or a modest increase in VPS26 paralogs, has no clear effect on retromer function, these results justify investigating the effect of VPS35 and VPS26 co-expression.

### Example 3— Results Using Neuroblastoma Cells and Plasmids and AAV9 Constructs

Neuroblastoma (N2A) cells were transfected with plasmids expressing single proteins (VPS35, VPS26a, or VPS26b), or a combination of proteins (VPS35+VPS26a or VPS35+VPS26b). A plasmid expressing GFP or an empty plasmid were used as controls.

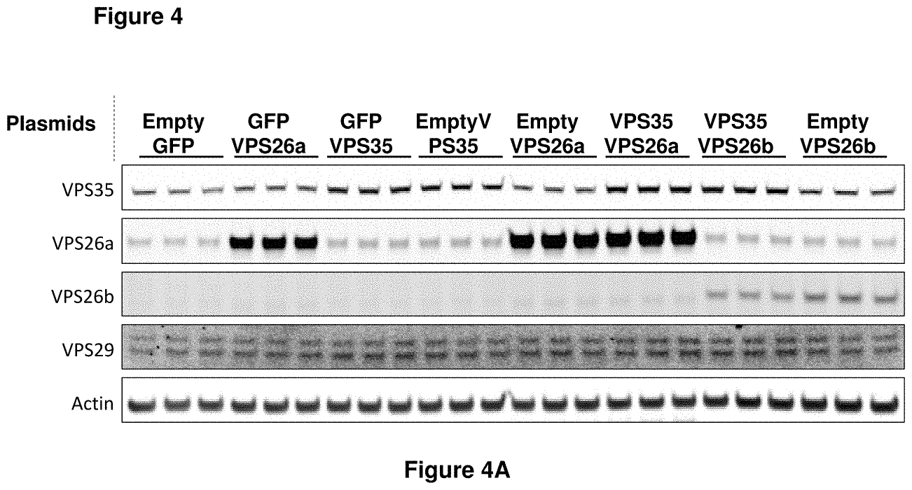

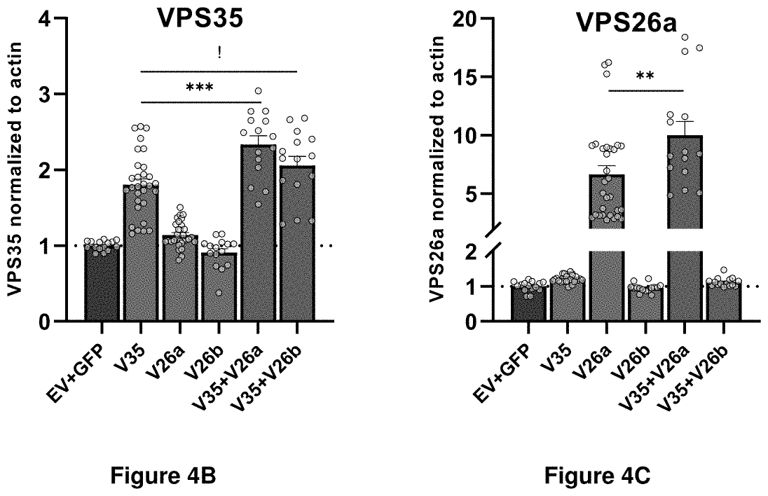

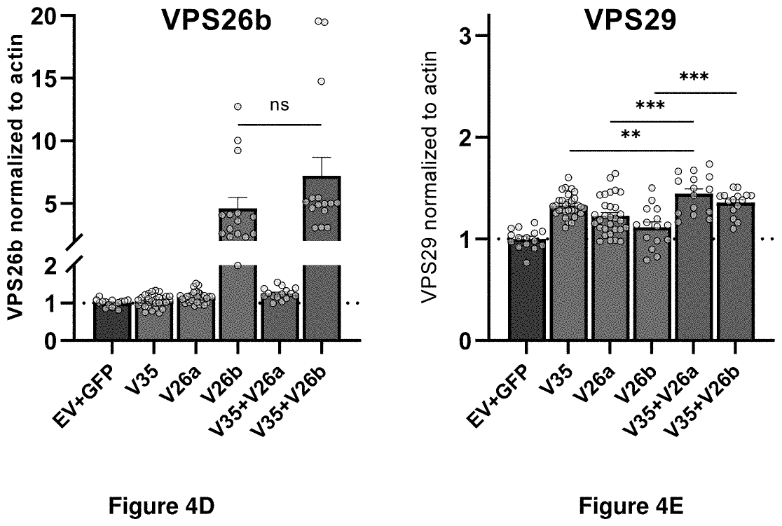

Single protein conditions resulted in overexpression of each protein above control levels: for VPS35 alone (80%, p=3.4E-09), for VPS26a alone (550%; p=2.2E-06), and for VPS26b alone (362%; p=0.0002). When compared to single protein conditions, VPS35+VPS26a expression resulted in a significant increase in VPS35 (31%; p=0.0003), VPS29 (17%; p=0.0007), and VPS26a (52%; p=0.015), but minimal change in VPS26b. VPS35+VPS26b expression resulted in a non-significant increase in VPS35 (15%; p=0.07), and VPS26b (56%; p=0.14), a significant increase in VPS29 (22%; p=0.0005), but no increase in VPS26a. See FIG. 4.

### Example 4—Results Using Neurons and AAV9 Constructs

To test the effect of VPS35 and VPS26 combinatorials in cultured neurons, five experimental AAV9 vectors, expressing mouse VPS35, VPS26a, VPS26b, and two control AAV9 vectors, one expressing GFP and the other an empty vector were generated. The experimental vectors were expressed in neurons in all possible combinations: Single protein expression (VPS35 alone, VPS26a alone, VPS26b alone); double protein expression (VPS35+VPS26a, VPS35+VPS26b, VPS26b+VPS26a); and triple protein expression (VPS35+VPS26a+VPS26b).

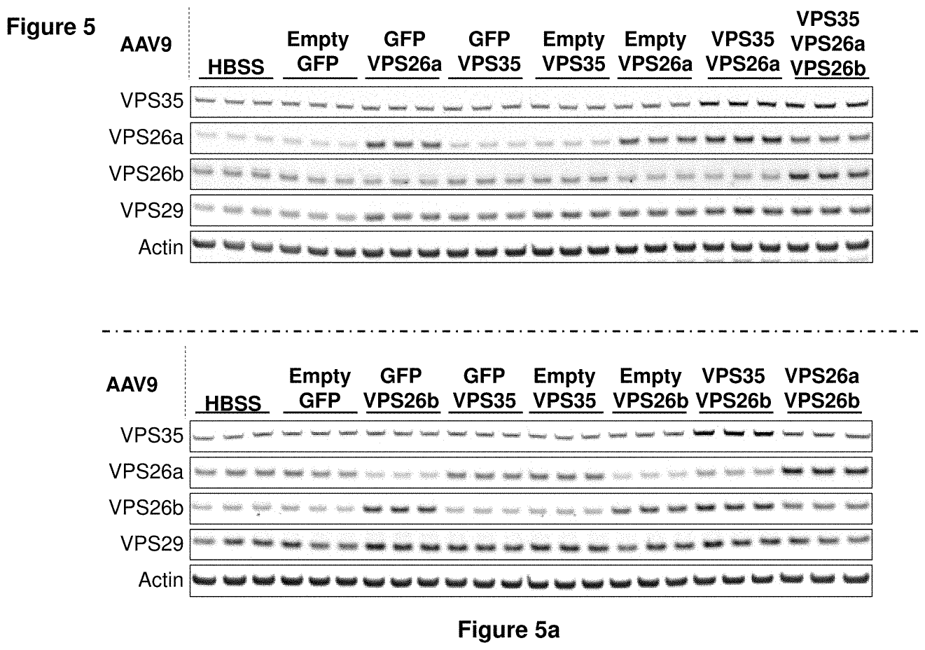

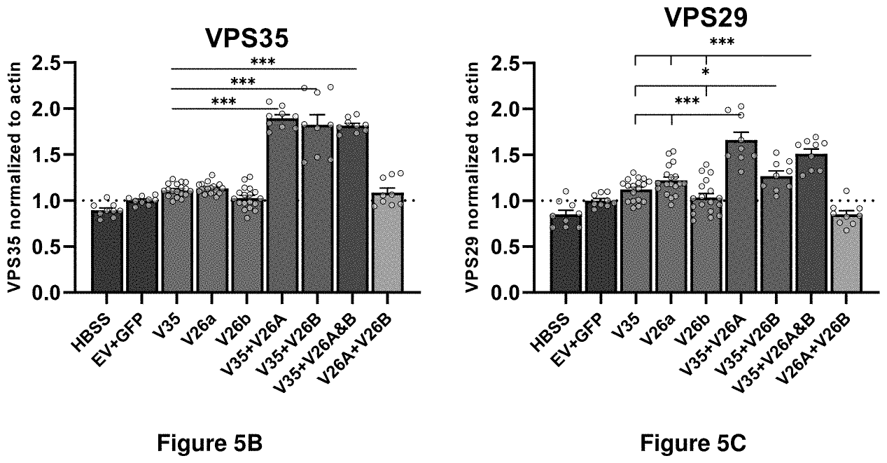

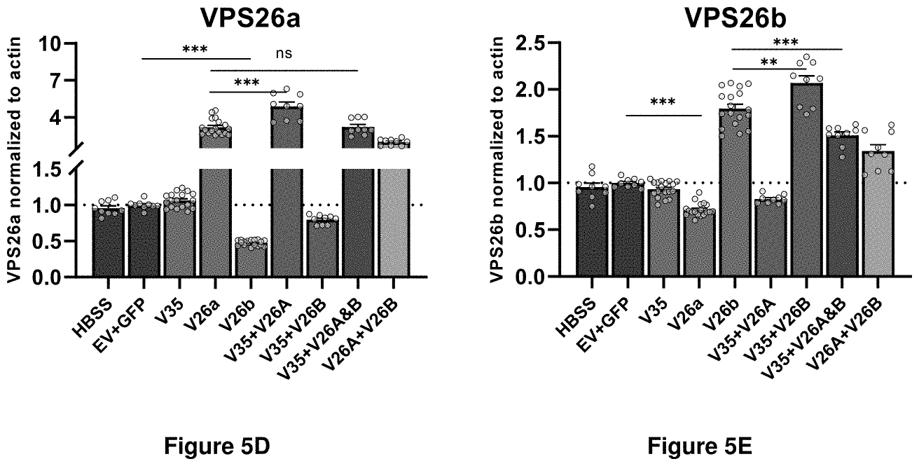

The dose of each viral vector was optimized in exploratory studies, and when used in the final combinatorial study, mean AAV9-VPS35 overexpression was 11% (range: 1% to 23%), mean AAV9-VPS26a was 218% (range: 154% to 354%) and mean AAV9-VPS26b was 80% (range: 50% to 107%) (FIG. 5B, blue bars). This profile turned out to be particularly useful for testing for interactions.

It was first tested whether there was VPS35-VPS26 interaction on retromer core protein expression, by comparing levels detected in single protein conditions to those detected in the combinatorial experiments. Compared to single protein expression, VPS35+VPS26a expression resulted in a significant increase in VPS35 (70%; p=4.4E-18), VPS26a (53%; p=2.1E-05), VPS29 (˜42%; p<1.22E-05), but not VPS26b. VPS35+VPS26b expression resulted in a significant increase in VPS35 (64%; p=3.6E-09), VPS26b (15%; p=0.003), VPS29 (˜18%; p<0.013), but not VPS26a.

Finally, VPS35+VPS26a+VPS26b expression resulted in a significant increase in all four retromer proteins compared to controls (EV+GFP)—VPS35 (81%; p=5.6E-15), VPS29 (51%; p<1.8E-07), VPS26a (220%; p=1.7E-08) and VPS26b (51%; p=9.8E-10).

See FIG. 5.

These results again documented a synergistic effect of the co-expression of VPS35+VPS26 on VPS35 expression, as well as on VPS26a and VPS26b.

While the main purpose of this comprehensive series of experiments was to test for synergistic interactions, the fact that VPS35+VPS26a had no effect on VPS26b, and that VPS35+VPS26b had no effect on VPS26a, suggests that in neurons each VPS26 paralog exists in biochemically distinct trimers.

### Example 5—Combined VPS35 and VPS26 Expression Synergizes Retromer Function in Neurons

Next it was tested whether VPS35 and VPS26 combinatorials have a synergistic effect on retromer function, by comparing levels of Sorll measured across all conditions because loss-of-function mutations in SORL1 are causal in Alzheimer's disease (Holstege et al. 2017), and an approximate 30% reduction in Sorll protein is found even in early stages of the sporadic disease (Sager et al. 2007); Scherzer et al. 2004; Dodson et al. 2006).

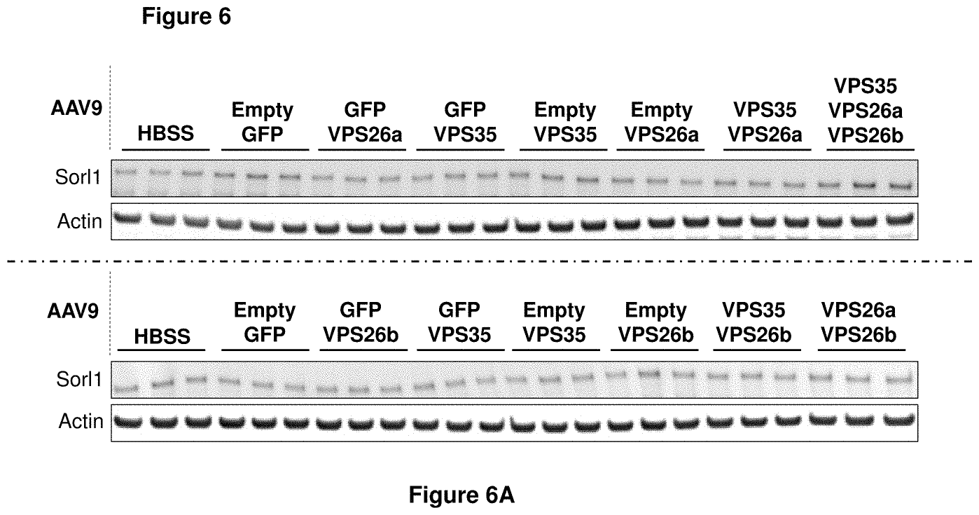

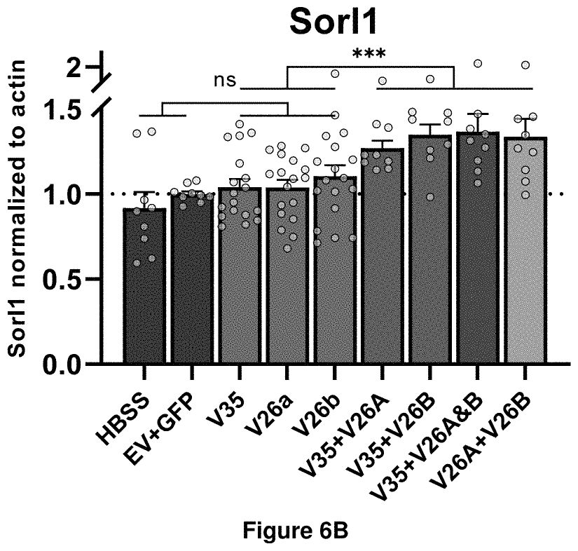

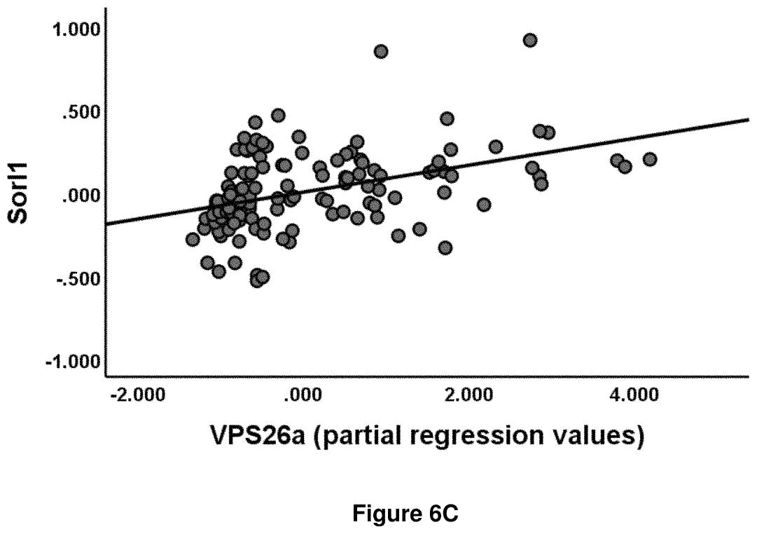

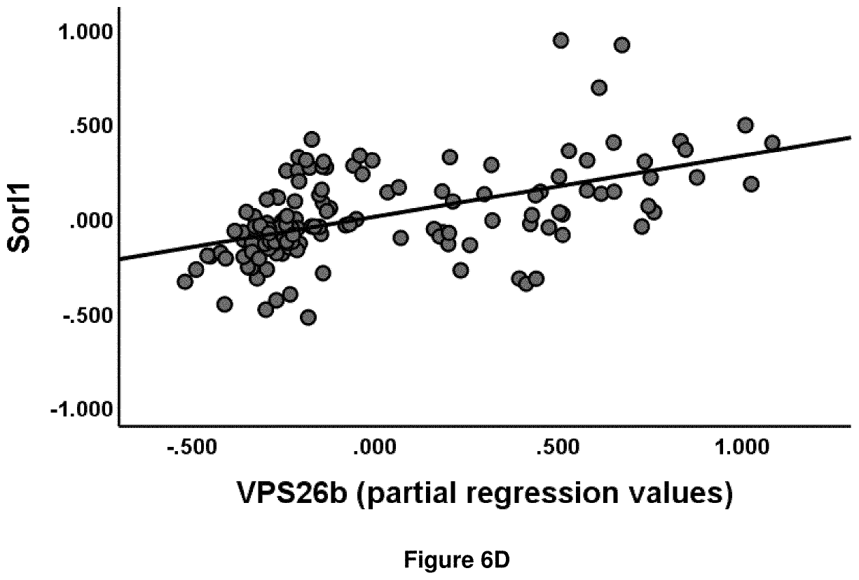

An univariate ANOVA was used, in which control conditions, single conditions, and combinatorial conditions were included as the fixed factor, and Sorll was included as the dependent variable. Results revealed a group effect (F=19.3, p=9.3E-8), with a simple comparison indicating that, while there was no difference between the control and single conditions (contrast estimate=0.1, p=0.9), there was a significant difference between the single and combination conditions (contrast estimate=0.4, p=2.2 E-7). Post-hoc comparisons revealed that each combination condition was significantly different than the single conditions (FIG. 6B).

Interestingly, a significant effect of VPS26a+VPS26b overexpression on Sorll (34%; p=0.006) (FIG. 6B) was also observed, even though this combination condition did not increase levels of VPS35 or VPS29 (FIG. 5B). Sorll has been found to interact with the retromer core via VPS26 (Suzuki et al. (2019), and this observation suggests that both paralogs might independently interact with Sorll.

The large-scale dataset that was generated, in which there are over 140 experimental or control conditions and in which the four retromer core proteins and Sorll were measured across a broad dynamic range were used. A multiple linear regression model was used, in which Sorll levels was included as the dependent variable, and VPS35, VPS26, VPS26a and VPS26b levels were simultaneously entered as the independent variables. A significant relationship to Sorll levels was found (F=19.5, p=1.1E-12), with only the VPS26 paralogs significantly contributing to the model. The model was therefore trimmed to include both paralogs, confirming that both VPS26a (t=5.6, p=1.4E-7) and VPS26b (F=7.2, p=3.2E-11) independently correlate with Sorll levels (FIGS. 6C and 6D). This result agreed with the interpretation that neurons have two trimers (VPS26b-VPS35-VPS29 and VPS26a-VPS35-VPS29) that are not only biochemically distinct, but functionally distinct as well.

### REFERENCES

Andersen, et al. Securing the future of drug discovery for central nervous system disorders. Nature reviews. Drug discovery. 2014; 13(12):871-872.
Bhalla et al. The location and trafficking routes of the neuronal retromer and its role in amyloid precursor protein transport. Neurobiol Dis. 2012; 47(1):126-34.
Collins et al. Structure of Vps26B and mapping of its interaction with the retromer protein complex. Traffic 2008; 9(3):366-79.
Dodson et al. LR11/SorLA expression is reduced in sporadic Alzheimer disease but not in familial Alzheimer disease. J Neuropathol Exp Neurol, 2006; 65(9): 866-72.
Eaton et al. Total protein analysis as a reliable loading control for quantitative fluorescent Western blotting. PloS one. 2013; 8(8): e72457.
Holstege et al. Characterization of pathogenic SORL1 genetic variants for association with Alzheimer's disease: a clinical interpretation strategy. Eur J Hum Genet. 2017.
Kendall et al. Mammalian Retromer Is an Adaptable Scaffold for Cargo Sorting from Endosomes. Structure. 2020; 28(4):393-405.e4.
Kirby et al. Adult hippocampal neural stem and progenitor cells regulate the neurogenic niche by secreting VEGF. PNAS USA. 2015; 112(13):4128-33. Epub 2015/03/17.
Qureshi et al. Retromer repletion with AAV9-VPS35 restores endosomal function in the mouse hippocampus. bioRxiv. 2019:618496.
Sager et al., Neuronal LR11/sorLA expression is reduced in mild cognitive impairment. Ann Neurol. 2007; 62(6):640-7.
Scherzer et al. Loss of apolipoprotein E receptor LR11 in Alzheimer disease. Arch Neurol. 2004; 61(8):1200-5.
Shi et al. The retromer subunit Vps26 has an arrestin fold and binds Vps35 through its C-terminal domain. Nat Struct Mol Biol. 2006; 13(6):540-8.
Small and Petsko Retromer in Alzheimer disease, Parkinson disease and other neurological disorders. Nature reviews. Neuroscience. 2015; 16(3):126-132.
Suzuki et al. A bipartite sorting signal ensures specificity of retromer complex in membrane protein recycling. J Cell Biol. 2019; 218(9):2876-2886.

1. A method of treating, preventing and/or curing a neurodegenerative disease or disorder in a subject in need thereof, comprising administering to the subject an effective amount of one or more viral vectors comprising a transgene encoding retromer core protein VPS35 and at least one transgene encoding a retromer core protein chosen from the group consisting of VPS26a, VPS26b, and combinations thereof.

2. A method of treating, preventing and/or curing a neurodegenerative disease or disorder in a subject in need thereof, comprising administering to the subject a therapeutically effective amount of one or more compositions comprising at least one viral vector comprising a transgene encoding retromer core protein VPS35 and at least one transgene encoding a retromer core protein chosen from the group consisting of VPS26a, VPS26b, and combinations thereof.

3. A method of treating, preventing and/or curing a neurodegenerative disease or disorder in a subject in need thereof, comprising administering to the subject a therapeutically effective amount of one or more compositions comprising a nucleic acid encoding retromer core protein VPS35 and at least one nucleic acid encoding a retromer core protein chosen from the group consisting of VPS26a, VPS26b, and combinations thereof.

4. A method of alleviating one or more symptoms of a neurodegenerative disease or disorder in a subject in need thereof, comprising administering to the subject an effective amount of the one or more viral vectors comprising a transgene encoding retromer core protein VPS35 and at least one transgene encoding a retromer core protein chosen from the group consisting of VPS26a, VPS26b, and combinations thereof.

5. A method of alleviating one or more symptoms of a neurodegenerative disease or disorder in a subject in need thereof, comprising administering to the subject a therapeutically effective amount of one or more compositions comprising at least one viral vector comprising a transgene encoding retromer core protein VPS35 and at least one transgene encoding a retromer core protein chosen from the group consisting of VPS26a, VPS26b, and combinations thereof.

6. A method of alleviating one or more symptoms of a neurodegenerative disease or disorder in a subject in need thereof, comprising administering to the subject a therapeutically effective amount of one or more compositions comprising a nucleic acid encoding retromer core protein VPS35 and at least nucleic acid encoding a retromer core protein chosen from the group consisting of VPS26a, VPS26b, and combinations thereof.

7. The method of any of claims 2 and 5, wherein a therapeutically effective amount of a first composition comprising at least one viral vector comprising a transgene encoding retromer core protein VPS35 is administered to a subject, and a therapeutically effective amount of a second composition comprising at least one viral vector comprising a transgene encoding a retromer core protein chosen from the group consisting of VPS26a, VPS26b, and combinations thereof is administered to the subject.

8. The method of claim 7, wherein a therapeutically effective amount of a third composition comprising at least one viral vector comprising a transgene encoding a retromer core protein chosen from the group consisting of VPS26a, VPS26b, and combinations thereof is administered to the subject.

9. The method of claim 7, wherein the first and second composition are administered sequentially.

10. The method of claim 7, wherein the first and second composition are administered simultaneously.

11. The method of claim 8, wherein the first, second and third composition are administered sequentially.

12. The method of claim 8, wherein the first, second and third composition are administered simultaneously.

13. The method of any of claims 2, 3, 5, and 6, wherein the composition further comprises a pharmaceutical carrier.

14. The method of any of claims 1-13, wherein the neurodegenerative disease or disorder is chosen from the group consisting of Alzheimer's disease (AD), Parkinson's disease, neuronal ceroid lipofuscinosis (NCL), transmissible spongiform encephalopathies (TSEs or prion disease), multiple system atrophy (MSA), progressive supranuclear palsy (PSP), frontotemporal lobar dementia linked to chromosome 17q21-22 and its subtypes (FTLD-17/FTLD-Tau), Lewy Body Disease (LBD), amyotrophic lateral sclerosis (ALS), frontal-temporal degeneration (FTD), ALS-FTD, and chronic traumatic encephalopathy (CTE).

15. A viral vector comprising at least one transgene encoding retromer core protein VPS35 and encoding a retromer core protein chosen from the group consisting of VPS26a, VPS26b, and combinations thereof.

16. The vector of claim 15, wherein the retromer core protein VPS35 encoded by the transgene has an amino acid sequence that is at least 85% identical to the amino acid sequence of VPS35 (SEQ ID NO: 1).

17. The vector of claim 15, wherein the retromer core protein VPS35 encoded by the transgene has the amino acid sequence of VPS35 (SEQ ID NO: 1).

18. The vector of claim 15, wherein the transgene encoding the retromer core protein Vps35 comprises human Vps35 (SEQ ID NO: 1).

19. The vector of claim 15, wherein the transgene has a nucleic acid sequence that is at least 70% identical to the nucleic acid sequence encoding VPS35 (SEQ ID NO: 1).

20. The vector of claim 15, wherein the transgene has the nucleic acid sequence of SEQ ID NO: 10.

21. The vector of claim 15, wherein the retromer core protein VPS26a encoded by the transgene has an amino acid sequence that is at least 85% identical to the amino acid sequence of VPS26a (SEQ ID NO: 2).

22. The vector of claim 15, wherein the retromer core protein Vps26a encoded by the transgene has the amino acid sequence of VPS26a (SEQ ID NO: 2).

23. The vector of claim 15, wherein the transgene encoding the retromer core protein Vps35 comprises human VPS26a (SEQ ID NO: 2).

24. The vector of claim 15, wherein the transgene has a nucleic acid sequence that is at least 70% identical to the nucleic acid sequence encoding VPS26a (SEQ ID NO: 2).

25. The vector of claim 15, wherein the retromer core protein VPS26b encoded by the transgene has an amino acid sequence that is at least 85% identical to the amino acid sequence of VPS26b (SEQ ID NO: 3).

26. The vector of claim 15, wherein the retromer core protein VPS26b encoded by the transgene has the amino acid sequence of VPS26b (SEQ ID NO: 3).

27. The vector of claim 15, wherein the transgene encoding the retromer core protein VPS35 comprises human VPS26b (SEQ ID NO: 3).

28. The vector of claim 15, wherein the transgene has a nucleic acid sequence that is at least 70% identical to the nucleic acid sequence encoding VPS26b (SEQ ID NO: 3).

29. The vector of any one of claims 1-28, wherein the vector is selected from the group consisting of adeno-associated virus (AAV), adenovirus, lentivirus, retrovirus, poxvirus, baculovirus, herpes simplex virus, vaccinia virus, and a synthetic virus.

30. The vector of claim 29, wherein the viral vector is an AAV.

31. The vector of claim 30, wherein the AAV is an AAV1, AAV2, AAV4, AAV5, AAV6, AAV7, AAV8, AAV9, AAV10, and AAVrh10.

32. The vector of claim 30, wherein the AAV is chosen from the group consisting of AAV9 and AAV2/9.

33. The vector of any one of claims 1-32, wherein the transgene is operably linked to a promoter that induces expression of the transgene in a neural cell.

34. The vector of any one of claims 1-33, wherein the transgene is operably linked to an enhancer that induces expression of the transgene in a neural cell.

35. A composition comprising at least one viral vector comprising a transgene encoding retromer core protein VPS35 and a transgene encoding a retromer core protein chosen from the group consisting of VPS26a, VPS26b, and combinations thereof.

36. The composition of claim 35, wherein the retromer core protein VPS35 encoded by the transgene has an amino acid sequence that is at least 85% identical to the amino acid sequence of VPS35 (SEQ ID NO: 1).

37. The composition of claim 35, wherein the retromer core protein VPS35 encoded by the transgene has the amino acid sequence of VPS35 (SEQ ID NO: 1).

38. The composition of claim 35, wherein the transgene encoding the retromer core protein VPS35 comprises human VPS35 (SEQ ID NO: 1).

39. The composition of claim 35, wherein the transgene has a nucleic acid sequence that is at least 70% identical to the nucleic acid sequence encoding VPS35 (SEQ ID NO: 1).

40. The composition of claim 35, wherein the transgene has the nucleic acid sequence of SEQ ID NO: 10.

41. The composition of claim 35, wherein the retromer core protein VPS26a encoded by the transgene has an amino acid sequence that is at least 85% identical to the amino acid sequence of VPS26a (SEQ ID NO: 2).

42. The composition of claim 35, wherein the retromer core protein Vps26a encoded by the transgene has the amino acid sequence of VPS26a (SEQ ID NO: 2).

43. The composition of claim 35, wherein the transgene encoding the retromer core protein Vps35 comprises human VPS26a (SEQ ID NO: 2).

44. The composition of claim 35, wherein the transgene has a nucleic acid sequence that is at least 70% identical to the nucleic acid sequence encoding VPS26a (SEQ ID NO: 2).

45. The composition of claim 35, wherein the retromer core protein VPS26b encoded by the transgene has an amino acid sequence that is at least 85% identical to the amino acid sequence of VPS26b (SEQ ID NO: 3).

46. The composition of claim 35, wherein the retromer core protein VPS26b encoded by the transgene has the amino acid sequence of VPS26b (SEQ ID NO: 3).

47. The composition of claim 35, wherein the transgene encoding the retromer core protein VPS35 comprises human VPS26b (SEQ ID NO: 3).

48. The composition of claim 35, wherein the transgene has a nucleic acid sequence that is at least 70% identical to the nucleic acid sequence encoding VPS26b (SEQ ID NO: 3).

49. The composition of claim 35, wherein the vector is selected from the group consisting of adeno-associated virus (AAV), adenovirus, lentivirus, retrovirus, poxvirus, baculovirus, herpes simplex virus, vaccinia virus, and a synthetic virus.

50. The composition of claim 49, wherein the viral vector is an AAV.

51. The composition of claim 50, wherein the AAV is an AAV1, AAV2, AAV4, AAV5, AAV6, AAV7, AAV8, AAV9, AAV10, and AAVrh10.

52. The composition of claim 50, wherein the AAV is chosen from the group consisting of AAV9 and AAV2/9.

53. The composition of any one of claims 35-52, wherein the transgene is operably linked to a promoter that induces expression of the transgene in a neural cell.

54. The composition of any one of claims 35-52, wherein the transgene is operably linked to an enhancer that induces expression of the transgene in a neural cell.

55. The composition of claim 35, further comprising a pharmaceutical carrier.

56. A composition comprising at least one nucleic acid encoding VPS35 and a retromer core protein chosen from the group consisting of VPS26a, VPS26b, and combinations thereof.

57. The composition of claim 56, comprising a first nucleic acid encoding VPS35 and a second nucleic acid encoding a retromer core protein chosen from the group consisting of VPS26a, VPS26b, and combinations thereof.

58. A kit comprising the composition of any of claims 35-57.
